(19) United States
(12) Patent Application Publication
(10) Pub. No.: US 2004/0181883 A1
(43) Pub. Date: Sep. 23, 2004

(54) Pasty anhydrous composition for simultaneously bleaching and dyeing human keratin fibers comprising at least one peroxygenated salt, at least one alkaline agent, at least one inert organic liquid and at least one cationic direct dye; process using such a compound; and kit comprising such a compound

(76) Inventors: Frederic Legrand, Courbevoie (FR); Jean-Marie Millequant, Saint Maur (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/740,432

(22) Filed: Dec. 22, 2003

Related U.S. Application Data
(60) Provisional application No. 60/468,633, filed on May 8, 2003.

(30) Foreign Application Priority Data
Dec. 20, 2002 (FR) 02 16404

(51) Int. Cl. A61K 7/13

(52) U.S. Cl. 8/405

(57) ABSTRACT

A pasty anhydrous composition for simultaneously bleaching and dyeing human keratin fibers, such as hair, comprising, in a medium that is suitable for dyeing:

at least one peroxygenated salt,

at least one alkaline agent,

at least one inert organic liquid present in an amount ranging from 15% to 35% by weight, relative to the total weight of the composition, and

at least one cationic direct dye. The at least one cationic dye may be chosen from azo dyes, azomethine dyes, methine dyes and xanthene dyes. A ready-to-use pasty anhydrous composition for simultaneously bleaching and dyeing human keratin fibers. A process of simultaneous bleaching and dyeing, and a device with compartments, or “kits”, comprising the described composition.

The inventors claim the benefit of U.S. Provisional Application No. 60/468,633, filed May 8, 2003.

Disclosed herein is an anhydrous composition, wherein the composition is in paste form, for simultaneously bleaching and dyeing human keratin fibers, such as hair, comprising at least one peroxygenated salt, at least one alkaline agent, at least one inert organic liquid and at least one cationic direct dye, and also to a process of simultaneous bleaching and dyeing.

Generally, in the hair field, two types of dyeing may be distinguished. The first is known as permanent dyeing or oxidation dyeing, which usually uses oxidation dyes that develop their dyeing power in the presence of oxidizing agents. The second is known as semi-permanent or temporary dyeing, or direct dyeing, which generally uses dyes capable of giving the hair a more or less pronounced change in color that, for example, can withstand shampooing several times. These dyes are known as direct dyes; they can be used with or without an oxidizing agent. In the presence of an oxidizing agent, the dyeing is known as lightening dyeing; without an oxidizing agent, it is known as conventional non-lightening direct dyeing.

The present disclosure relates to lightening direct dyeing, and therefore, is employed in the presence of an oxidizing agent, such as hyderogen peroxide.

It is a known practice to use ready-to-use lightening alkaline compositions comprising hydrogen peroxide and at least one direct dye to simultaneously lighten and color the hair.

Such compositions can usually result from mixing, prior to use of the composition, an aqueous hydrogen peroxide composition and an alkaline composition, wherein the alkaline composition is based on at least one direct dye.

However, this type of lightening performance of compositions remains limited, for instance, for applications on dark keratin fibers. Generally, bleaching compositions, wherein the composition is based on at least one peroxygenated salt, (such as persulphate) can be used to obtain greater lightening. The one-step process can consist of applying, to the area of the head of hair desired to be treated, a ready-to-use pulverulent composition for simultaneously bleaching and dyeing the hair, comprising hydrogen peroxide, at least one peroxygenated salt (for instance, persulphate), at least one alkaline agent and at least one direct dye. Such ready-to-use compositions are, for example, described in Patent Applications Nos. DE 19 721 785 and DE 19 721 797.

Similarly, Patent Application No. DE 3 814 685 describes ready-to-use compositions for simultaneously bleaching and dyeing the hair, consisting of mixing, before use, a pulverulent anhydrous bleaching composition comprising at least one peroxygenated salt, at least one alkaline agent, at least one direct dye and an aqueous hydrogen peroxide composition.

However, these compositions can have the drawback of being difficult to use, for instance, due to the volatility of the pulverulent bleaching composition.

Also, the hair bleaching compositions, most commonly used, are in the form of powders (mixtures) of small particle size, i.e. particles generally less than a millimeter, such as less than a few hundred microns in size, which can allow ready and rapid dissolution and/or dispersion in aqueous hydrogen peroxide solution. However, given their finely divided nature, such pulverulent compositions have several drawbacks: they are highly volatile and therefore give off harmful dusts during their handling; the materials of which these powders are composed (persulphates or alkaline silicates) are corrosive and irritant to the eyes, the respiratory pathways, and mucous membranes; and these powders are moreover difficult to handle and to measure out (problem of dusting and of flowability). Recently pastes comprising the pulverulent agents in a thickened inert organic liquid support have been developed. Such compositions are described, for instance, in Patent Applications Nos. DE 3 814 356 A1, DE 197 23 538 C1 and U.S. Pat. No. 4,170,637.

There is a real need for compositions that can simultaneously bleach and dye keratin fibers and that can overcome the volatility problems of pulverulent compositions.

Generally, simultaneous bleaching and dyeing compositions do not give very chromatic colorations, since the dyes can be destroyed by the powerful peroxygenated salts. Thus, the problem of stability and storage of the direct dyes in the composition arises.

The inventor has unexpectedly and advantageously, discovered, that the problem of the prior art compositions can be solved by use of a pasty anhydrous composition for simultaneously bleaching and dyeing human keratin fibers, such as hair, comprising, in a medium that is suitable for dyeing, at least one peroxygenated salt, at least one alkaline agent, at least one inert organic liquid and at least one cationic direct dye.

This paste can result in a composition is stable in storage and is capable of producing more chromatic and luminous colors. The colors obtained are vivid and have strong shades.

Disclosed herein is a pasty anhydrous composition for simultaneously bleaching and dyeing human keratin fibers, such as hair, comprising at least one peroxygenated salt, at least one alkaline agent, at least one inert organic liquid and at least one cationic direct dye.

Further disclosed herein is a ready-to-use composition for simultaneously bleaching and dyeing human keratin fibers, such as hair.

Disclosed herein is a process for simultaneously bleaching and dyeing human keratin fibers, such as hair, and also multi-compartment devices or “kits” comprising the composition.

Also disclosed herein is a pasty anhydrous composition for simultaneously bleaching and dyeing human keratin fibers, such as hair, comprising, in a medium that is suitable for dyeing:

at least one peroxygenated salt,

at least one alkaline agent,

at least one inert organic liquid present in an amount ranging from 15% to 35% by weight, relative to the total weight of the composition, and

at least one cationic direct dye.

For the purposes of the present disclosure, the term “pasty anhydrous composition” means a paste wherein the water content is less than 1% by weight relative to the total weight of the composition and, for instance, less than 0.5% by weight, relative to the total weight of the composition.

Also for the purposes of the present disclosure, the term “inert organic liquid” means a liquid that does not interact chemically with peroxygenated salts, or with the other constituents of the composition.

Further for the purposes of the present disclosure, the term “cationic direct dye” means a dye comprising at least one quaternized nitrogen atom.

The peroxygenated salts The at least one peroxygenated salt can be chosen from alkali metal and alkaline-earth metal persulphates, perborates, and percarbonates. For example, persulphates such as sodium persulphate and potassium persulphate can be used.

The peroxygenated salts, according to the disclosure, are present, for example, in an amount ranging from 10% to 70% and further for example, ranging from 20% to 60% of the total weight of the composition.

The alkaline agents The at least one alkaline agent is chosen from urea, ammonium salts, for instance, ammonium chloride, ammonium sulphate, ammonium phosphate, ammonium nitrate, silicates, phosphates and carbonates of alkali metals and alkaline-earth metals, such as lithium, sodium, potassium, magnesium, calcium, and barium.

The alkaline agents, as disclosed herein, are present, for example, in an amount ranging from 0.01% to 40% by weight, relative to the total weight of the composition, such as present in an amount ranging from 0.1% to 30% by weight, relative to the total weight of the composition.

The Inert Organic Liquid

According to the present disclosure, the at least one inert organic liquid may be chosen from groups formed by polydecenes of formula C₁₀ₙH_{[(20n)+2]} wherein n is an integer ranging from 3 to 9, esters of fatty alcohols and of fatty acids, esters and diesters of sugar of C₁₂-C₂₄ fatty acids, cyclic ethers and cyclic esters, silicone oils, mineral oils, and plant oils.

Polydecenes of Formula C₁₀ₙH_{[(20n)+2]} Wherein n is an Integer Ranging From 3 to 9

These compounds correspond to the name “polydecene” in the CTFA Dictionary 7th edition 1997 of the Cosmetic, Toiletry and Fragrance Association, USA, and also to the same INCI name in the USA and in Europe. The polydecenes, disclosed herein, are products formed from hydrogenation of poly-1-decenes. For example, these compounds, can be chosen from those wherein, n ranges from 3 to 7, in the formula above.

For example, the product sold under the name Silkflo® 366 NF Polydecene by the company Amoco Chemical, and those sold under the name Nexbase® 2002 FG, 2004 FG, 2006 FG and 2008 FG by the company Fortum.

As disclosed herein, the anhydrous bleaching and dyeing composition, comprises at least one polydecene present in an amount, for example, ranging from 15% to 35% by weight, relative to the total weight of the composition and further for example ranging from 15% to 25% by weight, relative to the total weight of the composition.

Esters of Fatty Alcohols and Fatty Acids

Among the esters of fatty alcohols and fatty acids that are used herein, non-limiting mention may be made of:

esters of saturated, linear and branched C₃-C₆ lower monoalcohols with monofunctional C₁₂-C₂₄ fatty acids (wherein the fatty acids can optionally be chosen from linear and branched, saturated and unsaturated fatty acids, for example, oleates, laurates, palmitates, myristates, behenates, cocoates, stearates, linoleates, linolenates, caprates and arachidonates, and mixtures thereof, such as oleo-palmitates, oleo-stearates, palmito-stearates, etc.). Among the esters, one can use, for example isopropyl palmitate and isopropyl myristate,

esters of linear and branched C₃-C₈ monoalcohols comprising bifunctional C₈-C₂₄ fatty acids (wherein the fatty acids may optionally be chosen from linear and branched, and saturated and unsaturated fatty acids), for instance, isopropyl diester of sebacic acid (such as diisopropyl sebacate),

esters of linear and branched C₃-C₈ monoalcohols comprising bifunctional C₂-C₈ fatty acids (wherein the fatty acids may optionally be chosen from linear and branched, and saturated and unsaturated fatty acids), for instance dioctyl adipate and dicaprylyl maleate,

esters of trifunctional acids, for instance triethyl citrate.

Esters and Diesters of Sugar of C₁₂-C₂₄ Fatty Acids

For purposes of the present disclosure, the term “sugar” means compounds comprising several alcohol functions, optionally comprising aldehyde and ketone functional groups, and comprising at least four carbon atoms. These sugars may, for example, be chosen from monosaccharides, oligosaccharides and polysaccharides.

As sugars that may be used according to the present disclosure, non-limiting mention may be made, for example, of sucrose, saccharose, glucose, galactose, ribose, fucose, maltose, fructose, mannose, arabinose, xylose and lactose, and derivatives thereof, for example alkyl derivatives, such as methyl derivatives, for instance methylglucose.

The esters of sugars and of fatty acids that may be used according to the present disclosure may be chosen, for example, from comprising esters and mixtures of esters of sugars described above and of linear and branched, saturated and unsaturated C₁₂-C₂₄ fatty acids.

As non-limiting examples, the esters may be chosen from mono-, di-, tri-, tetraesters and polyesters, and mixtures thereof.

The esters may be chosen for example from oleates, laurates, palmitates, myristates, behenates, cocoates, stearates, linoleates, linolenates, caprates and arachidonates, and mixtures thereof, for instance mixed oleo-palmitates, oleo-stearates, palmito-stearates, etc.

For instance, according to the present disclosure, the esters may also be chosen from mono- and diesters, for example, sucrose, glucose and methylglucose mono- and dioleates, stearates, behenates, oleopalmitates, linoleates, linolenates and oleostearates.

Non-limiting mention may be made of the product sold under the name Glucate DO by the company Amerchol, which is a methylglucose dioleate.

Additionally, non-limiting examples of esters and of mixtures of esters of sugar of fatty acid, may include:

the products sold under the names F160, F140, F110, F90, F70 and SL40 by the company Crodesta, respectively chosen from saccharose palmito-stearates formed from 73% monoester and 27% diester and triester, 61% monoester and 39% diester, triester and tetraester, 52% monoester and 48% diester, triester and tetraester, 45% monoester and 55% diester, triester and tetraester, and 39% monoester and 61% diester, triester and tetraester, and sucrose monolaurate;

the products sold under the name Ryoto Sugar Esters, for example referenced B370 and corresponding to saccharose behenate formed from 20% monoester and 80% di- triester-polyester;

sucrose mono-di- palmito-stearate sold by the company Goldschmidt under the name Tegosoft PSE.

Cyclic Ethers and Cyclic Esters

According to the present disclosure, the cyclic ethers and cyclic esters may be chosen, for example, from γ-butyrolactone, dimethyl isosorbide and diisopropyl isosorbide.

Silicone Oils

For purposes of the present disclosure, silicone oils are liquid, non-volatile silicone fluids comprising a viscosity less than or equal to 10 000 mPa.s at 25° C., the viscosity of the silicones being measured according to ASTM standard 445 Appendix C.

Silicone oils are defined in greater detail in Walter Noll's “Chemistry and Technology of Silicones” (1968)—Academic Press.

Among the silicone oils that may be used, as disclosed herein non-limiting examples that may be mentioned include silicone oils sold under the names DC-200 Fluid-5 mPa.s, DC-200 Fluid-20 mPa.s, DC-200 Fluid-350 mPa.s, DC-200 Fluid-1000 mPa.s, and DC-200 Fluid-10 000 mPa.s by the company Dow Corning.

Mineral Oils

Among the mineral oils that non-limiting mention may be made of include liquid paraffin.

Plant Oils

Among the plant oils that non-limiting mention may be made of are avocado oil, olive oil, and liquid jojoba wax.

For example, according to the present disclosure, at least one inert organic liquid can be chosen from polydecenes and esters of fatty alcohols and fatty acids.

As disclosed herein, the at least one inert organic liquid is, for example, present in an amount ranging from 15% to 35% by weight, relative to the total weight of the paste.

In one aspect of the disclosure, the compositions that allow the production of the most stable colors are obtained, for example, by using cationic direct dyes chosen from xanthene dyes, azo dyes, azomethine dyes, and methine dyes.

For instance, heterocyclic cationic direct dyes can be used, and this dye can comprise at least one cationic charge on a heterocycle.

For example, direct dyes comprising at least one cationic charge on a heterocycle should be used.

Further, for example, azo dyes, methine dyes, and azomethine dyes comprising at least one cationic charge on a heterocycle should be used.

According to the present disclosure, the at least one cationic direct dye can be chosen from the following dyes:

cationic xanthene dyes, for instance Acid Red 52,

cationic azo and azomethine direct dyes, such as Basic Blue 41, Basic Blue 67, Basic Brown 1, Basic Brown 4, Basic Red 18, Basic Red 22, Basic Red 46, Basic Red 104, Basic Violet 35, Basic Yellow 45, Basic Yellow 57 and Basic Yellow 67,

cationic methine direct dyes, such as Basic Red 14, Basic Yellow 13 and Basic Yellow 29,

and the dyes described in Patent Application No. EP 1 025 834:

of formula (I)

G-N=N-J (I)

wherein:

G is chosen from G₁, G₂ and G₃ below:

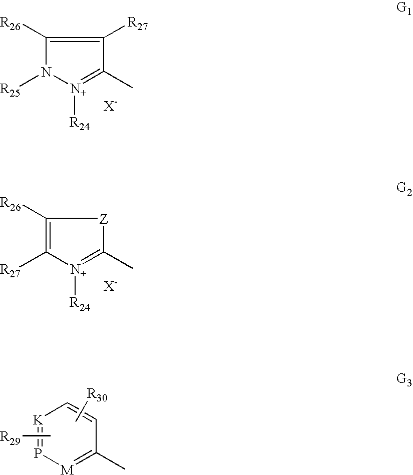

wherein:

R₂₄ is chosen from C₁-C₄ alkyl, phenyl which may be substituted with C₁-C₄ alkyl, and halogen atoms chosen from chlorine, bromine, iodine and fluorine;

R₂₅ is chosen from C₁-C₄ alkyl and phenyl;

R₂₆ and R₂₇, which may be identical or different, are chosen from C₁-C₄ alkyl and phenyl, and additionally wherein R₂₆ and R₂₇ may form together in G₁ a benzene ring comprising at least one substituent chosen from C₁-C₄ alkyl, C₁-C₄ alkoxy, and NO₂, and also wherein R₂₆ and R₂₇ may form together in G₂ a benzene ring optionally comprising at least one substituent chosen from C₁-C₄ alkyl, C₁-C₄ alkoxy, and NO₂;

R₂₆ may also be chosen from hydrogen;

Z is chosen from oxygen, sulphur and groups of formula —NR₂₅;

M is chosen —CH, —CR (wherein R comprises C₁-C₄ alkyl), and —N R₂₈(X⁻)ᵣ;

K is chosen from —CH, —CR (wherein R comprises C₁-C₄ alkyl), and —NR₂₈(X⁻)ᵣ;

P is chosen from —CH, —CR (wherein R comprises C₁-C₄ alkyl), and —NR₂₈(X⁻)ᵣ;

wherein r is an integer chosen from 0 and 1;

R₂₈ is chosen from O⁻, C₁-C₄ alkoxy, and C₁-C₄ alkyl;

R₂₉ and R₃₀, which may be identical or different, are chosen from hydrogen and halogen atoms chosen from chlorine, bromine, iodine and fluorine, C₁-C₄ alkyl, C₁-C₄ alkoxy, and —NO₂;

X⁻ is chosen from anions, such as chloride, iodide, methyl sulphate, ethyl sulphate, acetate and perchlorate anions;

J represents:

(a) J₁ below:

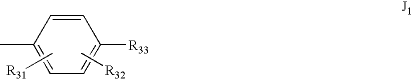

wherein:

R₃₁ is chosen from hydrogen, halogen atoms chosen from chlorine, bromine, iodine and fluorine, C₁-C₄ alkyl, C₁-C₄ alkoxy, —OH, —NO₂, —NHR₃₄, —NR₃₅R₃₆, and C₁-C₄-NHCOalkyl, and wherein R₃₁ may form with R₃₂, at least one ring chosen from 5- and 6-membered rings optionally comprising at least one hetero atom chosen from nitrogen, oxygen and sulphur;

R₃₂ is chosen from hydrogen, halogen atoms chosen from chlorine, bromine, iodine and fluorine, C₁-C₄, C₁-C₄ alkoxy, and wherein R₃₂ may form with R₃₃ and R₃₄, at least one ring chosen from 5- and 6-membered rings optionally comprising at least one hetero atom chosen from nitrogen, oxygen and sulphur;

R₃₃ is chosen from hydrogen, —OH, —NHR₃₄ and —NR₃₅R₃₆;

R₃₄ is chosen from hydrogen, C₁-C₄ alkyl, C₁-C₄ monohydroxyalkyl, C₂-C₄ polyhydroxyalkyl, and phenyl;

R₃₅ and R₃₆, which may be identical or different, are chosen from C₁-C₄ alkyl, C₁-C₄ monohydroxyalkyl, and C₂-C₄ polyhydroxyalkyl;

(b) 5- and 6-membered nitrogenous heterocyclic groups, optionally comprising other hetero atoms and carbonyl groups and optionally comprising at least one substituent chosen from C₁-C₄ alkyl, amino, and phenyl, such as groups of formula J₂ below:

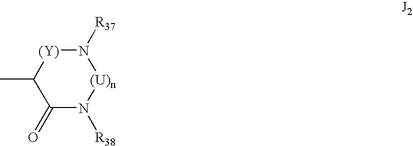

wherein:

R₃₇ and R₃₈, which may be identical or different, are chosen from hydrogen, C₃-C₁₀ alkyl, and phenyl;

Y is chosen from —CO— and —C(CH₃)═;

n is an integer chosen from 0 and 1, wherein when n is equal to 1,U is chosen from —CO—,

compounds of formula (II) below:

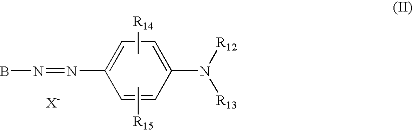

wherein:

wherein:

R₁₂ is chosen from hydrogen and C₁-C₄ alkyl,

R₁₃ is chosen from hydrogen, alkyl optionally having at least one substituent chosen from —CN, amino, ;and 4′-aminophenyl, and wherein R₁₃ may form with R₁₂ a heterocycle optionally comprising at least one heteroatom chosen from oxygen and nitrogen, wherein the heterocycle may optionally have at least one substituent chosen from C₁-C₄ alkyl,

R₁₄ and R₁₅, which may be identical or different, are chosen from hydrogen, halogen atoms chosen from bromine, chlorine, iodine and fluorine, C₁-C₄ alkyl, C₁-C₄ alkoxy, and —CN,

X⁻ is chosen from anions, such as chloride, methyl sulphate and acetate anions,

B is chosen from B1 to B6 below:

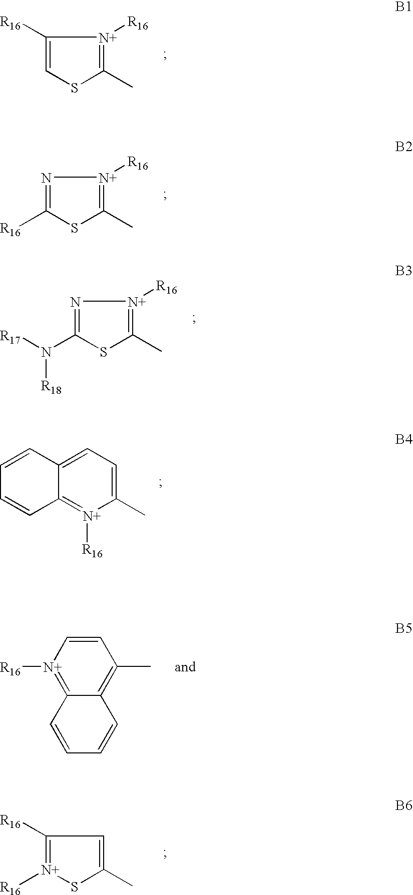

wherein R₁₆ , which may be identical or different, is chosen from C₁-C₄ alkyl,

R₁₇ and R₁₈, which may be identical or different, are chosen from hydrogen and C₁-C₄ alkyl,

compounds of formulae (III) and (IV) below:

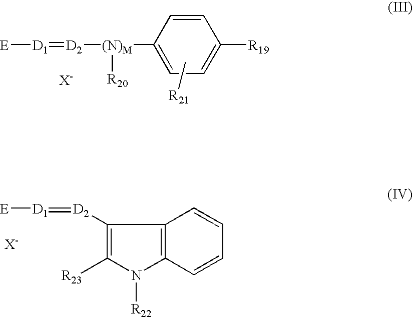

wherein:

R₁₉ is chosen from hydrogen, C₁-C₄, halogen, and amino,

R₂₀ is chosen from hydrogen and C₁-C₄ alkyl, and wherein R₂₀ may form, with a carbon atom of the benzene ring, a heterocycle optionally comprising oxygen and optionally having at least one substituent chosen from C₁-C₄ alkyl,

R₂₁ is chosen from hydrogen and halogen atom,

R₂₂ and R₂₃, which may be identical or different, are chosen from hydrogen and C₁-C₄ alkyl,

D₁ and D₂, which may be identical or different, are chosen from nitrogen and —CH,

m is an integer chosen from 0 and 1,

wherein when R₁₉ is chosen from unsubstituted amino groups, then D₁ and D₂, which may be identical or different, are chosen from —CH and m is an integer equal to 0,

X⁻ is chosen from anions, such as chloride, methyl sulphate, and acetate anions,

E is chosen from E1 to E8 below:

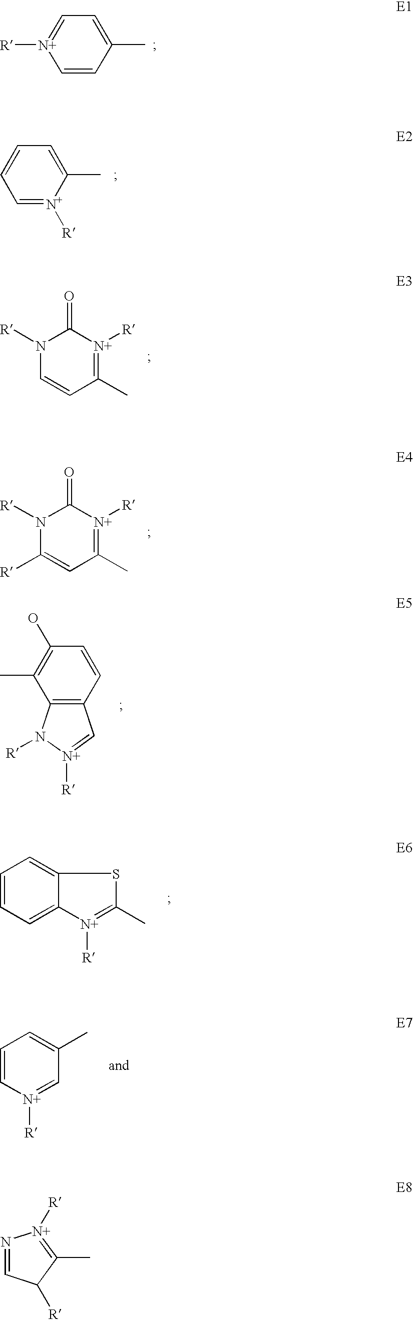

wherein R′ is chosen from C₁-C₄ alkyl;

wherein when m is an integer equal to 0 and D₁ is chosen from nitrogen, then E may be chosen from E9 below:

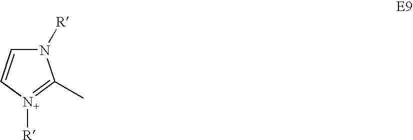

wherein R′ is chosen from C₁-C₄ alkyl

compounds of formula (V) below:

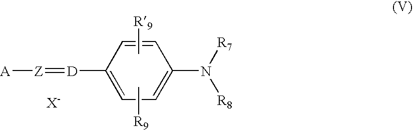

wherein:

Z and D, which may be identical or different, can be chosen from nitrogen and —CH,

R₇ and R₈, which may be identical or different, can be chosen from hydrogen; C₁-C₄ alkyl optionally having at least one substituent chosen from —CN, —OH, and —NH₂ radicals, and wherein R₇ and R₈ can form, with a carbon atom of the benzene ring, heterocycles optionally comprising oxygen and an additional nitrogen, optionally having at least one substituent chosen from C₁-C₄ alkyl radical and 4′-aminophenyl,

R₉ and R′₉, which may be identical or different, are chosen from hydrogen and halogen chosen from chlorine, bromine, iodine, and fluorine, cyano, C₁-C₄ alkyl, C₁-C₄ alkoxy and acetyloxy,

X⁻ is chosen from anions, such as chloride, methyl sulphate and acetate anions,

A is chosen from A1 to A19 below:

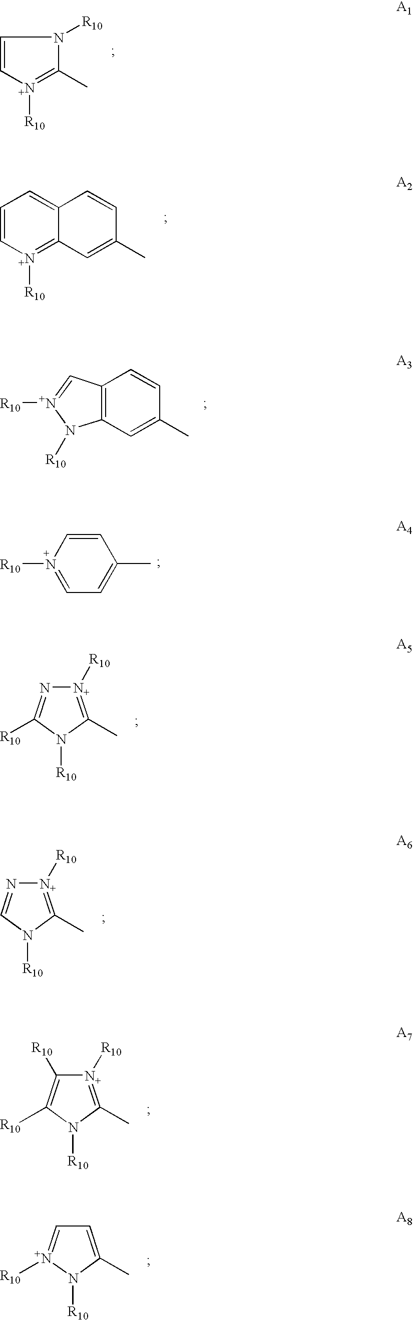

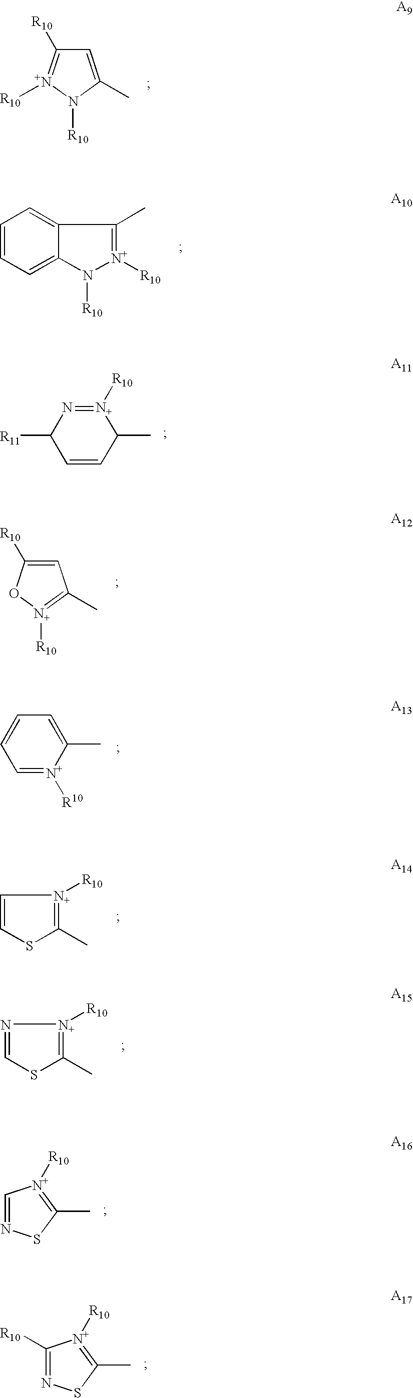

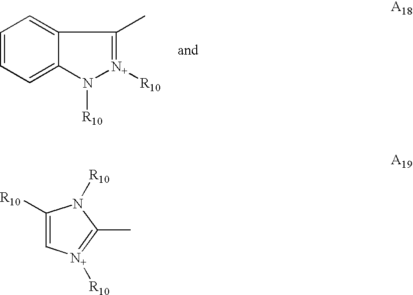

wherein R₁₀, which may be identical or different, is chosen from C₁-C₄ alkyl optionally having at least one hydroxyl substituent, and R₁₁ is chosen from C₁-C₄ alkoxy,

the dyes described in Patent Application No. EP 714 954, of formulae:

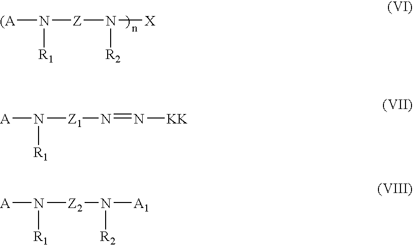

wherein:

A and A₁, which may be identical or different, are chosen from residues of formula:

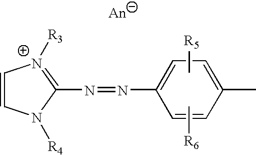

Z is chosen from aliphatic and aromatic diamine residues,

R₁ and R₂, which may be identical or different, are chosen from hydrogen, and C₁-C₄ alkyl, and wherein R₁ and R₂ may form, together with two nitrogen atoms to which they are attached or with Z and Z₂, a ring chosen from 5-, 6- and 7-membered rings,

X is chosen from residues of a chain unit forming a bridge,

n is an integer chosen from 2, 3 and 4,

Z₁ is chosen from residues of aromatic diamines,

Z₂ is chosen from residues of aliphatic diamines,

KK is chosen from residues of a coupling compound,

R₃ and R₄, which may be identical or different, are chosen from hydrogen and C₁-C₄ alkyl,

R₅ and R₆, which may be identical or different, are chosen from hydrogen, C₁-C₄ alkyl, and C₁-C₄ alkoxy,

An^{Θ} is chosen from colorless anions,

and cationic dyes described in Patent Applications Nos. WO 95/01 772, WO 95/15 144, EP 714 954, EP 1 170 000, EP 1 166 753, EP 1 166 754 and EP 1 170 001, which are different from the above dyes. The passages in these patent applications that relate to cationic dyes are incorporated herein by reference.

For example, the dyes that can be used are Basic Red 51 of formula (IX):

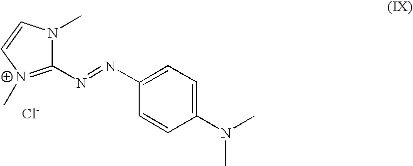

Basic Yellow 87 of formula (X):

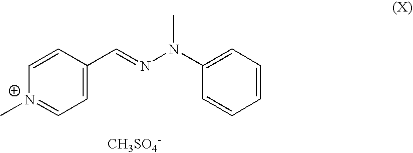

and Basic Orange 31 of formula (XI):

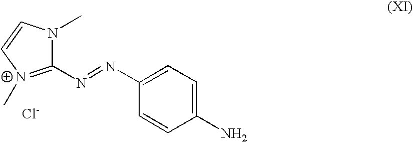

The cationic direct dyes, as disclosed herein, can be present in an amount ranging from 0.001% to 20% by weight, relative to the total weight of the composition, such as in an amount ranging from 0.01% to 10% by weight, relative to the total weight of the composition and, for example, in an amount ranging from 0.1% to 5% by weight, relative to the total weight of the composition.

These compositions may comprise at least one amphiphilic polymer, which can, for example, be nonionic and/or anionic, comprising at least one fatty chain. Generally, since these traditional thickeners, can over time, bring about a drop in the viscosity of bleaching compositions, the Inventor has, in French Patent No. 2 788 974, proposed using a thickening system capable of maintaining a high viscosity for the time required to obtain the desired bleaching, and comprising combining conventional water-soluble thickeners with nonionic amphiphilic polymers comprising at least one fatty chain.

Moreover, since bleaching treatments can generally be aggressive and may result in poor cosmetic properties of the hair, such as difficult disentangling, an unpleasant feel or coarse, dull hair, and, for example, degradation of the fibers, the Inventor has, in French Patent No. 2 788 976, proposed to limit this degradation by using a combination of nonionic and/or anionic amphiphilic polymers and of cationic and amphoteric substantive polymers.

Nonionic Amphiphilic Polymers Comprising at Least One Fatty Chain

The nonionic amphiphilic polymers comprising at least one fatty chain can be chosen, for example, from:

(1) celluloses modified with groups comprising at least one fatty chain; non-limiting mention may be made, for example, of:

hydroxyethylcelluloses modified with groups comprising at least one fatty chain, such as alkyl, arylalkyl and alkylaryl groups, and mixtures thereof, and wherein the alkyl groups are chosen, for example from C₈-C₂₂ groups, such as the product Natrosol Plus Grade 330 CS® (C₁₆ alkyls) sold by the company Aqualon, and the product Bermocoll EHM 100 sold by the company Berol Nobel,

hydroxyethylcelluloses modified with polyalkylene glycol alkylphenol ether groups, such as the product Amercell Polymer HM-1500® (polyethylene glycol (15) nonylphenol ether) sold by the company Amerchol.

(2) hydroxypropyl guars modified with groups comprising at least one C₈ to C₂₂ fatty chain, such as the product Jaguar® XC-95/3 (C₁₄ alkyl chain) sold by the company Rhodia, the product Esaflor® HM 22 (C₂₂ alkyl chain) sold by the company Lamberti, and the products RE210-18® (C₁₄ alkyl chain) and RE205-1® (C₂₀ alkyl chain) sold by the company Rhône-Poulenc.

(3) copolymers of vinylpyrrolidone and of hydrophobic monomers comprising fatty chains, non-limiting mention may be made, for example, of:

products Antarone V216 or Ganex® V216 (vinylpyrrolidone/hexadecene copolymer) sold by the company I.S.P.,

products Antaron® V220 or Ganex® V220 (vinylpyrrolidone/eicosene copolymer) sold by the company I.S.P.

(4) copolymers of C₁-C₆ alkyl acrylates and methacrylates and of amphiphilic monomers comprising at least one fatty chain.

(5) copolymers of hydrophilic acrylates and methacrylates and of hydrophobic monomers comprising at least one fatty chain, such as polyethylene glycol methacrylate/lauryl methacrylate copolymers.

(6) polymers with an aminoplast ether skeleton comprising at least one fatty chain, such as Pure Thix® compounds sold by the company Sud-Chemie.

(7) polyurethane polyethers comprising hydrophilic blocks, such as of polyoxyethylenated nature, and hydrophobic blocks that may be comprised of aliphatic chains, cycloaliphatic and aromatic chains.

For example, the polyurethane polyethers can comprise at least two hydrocarbon-based lipophilic chains, comprising from 6 to 30 carbon atoms, separated by a hydrophilic block, wherein the hydrocarbon-based chains may optionally be chosen from pendent chains and chains at the end of a hydrophilic block. For example, it is possible for at least one pendent chain to be provided. In addition, the polymer may comprise hydrocarbon-based chains optionally at one or both ends of a hydrophilic block.

The polyurethane polyethers may be chosen from multiblock forms, such as triblock form. The hydrophobic blocks may be found at each end of the chain (for example, triblock copolymers comprising a hydrophilic central block) or distributed at both ends and in the chain (for example, multiblock copolymers). These polymers may also be chosen from graft polymers and starburst polymers.

The fatty-chain nonionic polyurethane polyethers may be chosen from triblock copolymers, wherein the hydrophilic block is chosen from polyoxyethylenated chains comprising from 50 to 1000 oxyethylenated groups. The nonionic polyurethane polyethers can comprise urethane bonds between the hydrophilic blocks, hence the name of the compound.

Further, the fatty-chain nonionic polyurethane polyethers can be chosen from polymers wherein the hydrophilic blocks are linked to the lipophilic blocks via other chemical bonds.

As examples of fatty-chain nonionic polyurethane polyethers that may be used, non-limiting mention may be made of Ser-Ad FX 1100® from the company Servo Delden, which is a copolymer known under the European and US INCI name “Steareth-100/PEG-136/HMDI Copolymer”.

Rheolate® 205 comprising a urea function, sold by the company Rheox, or Rheolates® 208, 204 or 212 or Acrysol® RM 184, may also be used.

Mention may also be made of the product Elfacos® T210 comprising C₁₂-₁₄ alkyl chains and the product Elfacos® T212 comprising C₁₈ alkyl chains, from Akzo.

The polyurethane polyethers that may be used, as disclosed herein include, for example, those described in the article by G. Fonnum, J. Bakke and Fk. Hansen—Colloid Polym. Sci. 271, 380-389 (1993).

Polyurethane polyethers comprising at least one C₁₀ to C₂₀ fatty chain, and hydroxypropyl guars modified with groups comprising at least one C₈ to C₂₂ fatty chain, are, for instance, used.

Anionic Amphiphilic Polymers Comprising at Least One Fatty Chain

According to the present disclosure, the anionic amphiphilic polymers comprising at least one fatty chain can optionally be crosslinked polymers comprising:

hydrophilic units derived from at least one monomer comprising ethylenic unsaturated hydrocarbons comprising groups chosen from free carboxylic acid functional groups, and free, partially, and totally neutralized sulphonic functional groups, and

hydrophobic units derived from at least one monomer comprising ethylenic unsaturated hydrocarbons comprising hydrophobic side chains, and optionally comprising

crosslinking units derived from at least one polyunsaturated monomer.

(I) The monomers comprising ethylenic unsaturated hydrocarbons comprising carboxylic acid functional groups can be chosen from ethacrylic acids, methacrylic acids and acrylic acids, such as methacrylic acids and acrylic acids and mixtures thereof.

The monomers comprising ethylenic unsaturated hydrocarbons comprising hydrophobic side chains can be chosen from (i) esters of unsaturated carboxylic acids and fatty alcohols and (ii) ethers of allyl and fatty alcohols.

(i) The fatty alcohols esters of unsaturated carboxylic acids are chosen, for example, from C₁₀-C₃₀, such as C₁₂-C₂₂, alkyl ethacrylates, methacrylates and acrylates. The fatty alcohols esters of unsaturated carboxylic acids can be chosen, for example, from lauryl acrylates, stearyl acrylates, decyl acrylates, isodecyl acrylates and dodecyl acrylates, as well as the corresponding methacrylates, i.e. lauryl methacrylates, stearyl methacrylates, decyl methacrylates, isodecyl methacrylates and dodecyl methacrylates.

(ii) The allyl fatty alcohols ethers forming the hydrophobic units of the anionic amphiphilic polymers, as disclosed herein, correspond to the following formula (1):

CH₂═C R′ CH₂ O Bₙ R (1)

wherein R′ is chosen from hydrogen and CH₃, B is chosen from ethylenoxy radicals, n is an integer ranging from 0 to 100, R is a hydrocarbon-based group chosen from alkyl, arylalkyl, aryl, alkylaryl and cycloalkyl radicals comprising from 8 to 30 carbon atoms, such as from 10 to 24 carbon atoms, and, for example, from 12 to 18 carbon atoms. For example, one unit of formula (I) which can be used is a unit wherein R′ is chosen from hydrogen, n is equal to 10 and R is chosen from a stearyl (C₁₈) radical.

The crosslinking monomer can be chosen from a compound comprising at least two non-conjugated polymerizable double bonds. Non-limiting examples that may be mentioned are diallyl phthalate, allyl (meth)acrylate, divinylbenzene, (poly)ethylene glycol dimethacrylate, methylenebisacrylamide, polyallylsucrose, and polyallylpentaerythritol.

Anionic amphiphilic polymers, as disclosed herein, are described and prepared, for example, in U.S. Pat. Nos. 3,915,921 and 4,509,949 (disclosing copolymers of (meth)acrylic acid and of C₁₀-C₃₀ alkyl (meth)acrylates), or in Patent No. EP-0 216 479 B2 (disclosing copolymers of (meth)acrylic acid and of fatty alcohol allyl ethers).

Non-limiting examples of polymers that may be mentioned are:

crosslinked polymers of acrylic acid and of C₁₀-C₃₀ alkyl methacrylate, such as Carbopol ETD 2020 sold by the company Goodrich;

crosslinked polymers of acrylic acid and of C₁₀-C₃₀ alkyl acrylate, such as the polymers sold under the names Carbopol® 1382, Pemulen® TR1 and Pemulene TR2 by the company Goodrich;

methacrylic acid/ethyl acrylate/oxyethylenated stearyl methacrylate (55/35/10) terpolymer;

(meth)acrylic acid/ethyl acrylate/25 EO oxyethylenated behenyl methacrylate terpolymer, and

methacrylic acid/ethyl acrylate/steareth-10 allyl ether crosslinked terpolymer.

(II) The amphiphilic polymers comprising as hydrophilic units at least one ethylenically unsaturated monomer comprising a sulphonic group, existing in forms chosen from free, partially and totally neutralized forms, and at least one hydrophobic portion, are described, for example, in the Inventor's French Patent Applications Nos. 0 016 954 and 0 100 328 by, the content of which are incorporated, by reference, herein.

Among these polymers, non-limiting mention may be made, for example, of:

2-acrylamido-2-methylpropanesulphonic acid (AMPS)/n-dodecylacrylamide copolymers neutralized with sodium hydroxide, copolymers crosslinked with methylenebisacrylamide comprising of 75% by weight of AMPS units neutralized with NH₃ and of 25% by weight of acrylate units of Genapol® T-250, copolymers crosslinked with allyl methacrylate comprising of 90% by weight of AMPS units neutralized with NH₃ and of 10% by weight of methacrylate units of Genapol® T-250, and copolymers crosslinked with allyl methacrylate comprising of 80% by weight of AMPS units neutralized with NH₃ and of 20% by weight of methacrylate units of Genapol® T-250.

In the anhydrous pasty composition for simultaneous bleaching and dyeing, as disclosed herein, the at least one nonionic and anionic amphiphilic polymer comprising at least one fatty chain may be present, for example, in an amount ranging from 0.01% to 30% by weight, relative to the total weight of the bleaching powder, and, for example, in an amount ranging from 0.01% to 15% by weight, relative to the total weight of the bleaching powder.

According to the present disclosure, the anhydrous pasty composition may also comprise anhydrous cationic and amphoteric conditioning polymers that are well known to those of ordinary skill in the art and that are described in French Patents Nos 2 788 974 and 2 788 976 and described below.

Cationic Polymers

For the purposes of this disclosure, the expression “cationic polymer” means any polymer comprising cationic groups and/or groups which may be ionized into cationic groups.

The cationic polymers which can be, as disclosed herein, may be chosen from any of those already known by those skilled in the art as improving the cosmetic properties of the hair, for example, those described in Patent Application No. EP-A-337 354 and in French Patents Nos. FR-2 270 846, 2 383 660, 2 598 611, 2 470 596 and 2 519 863.

The cationic polymers may, for example, be chosen from those comprising units comprising at least one amine group chosen from primary, secondary, tertiary and quaternary amine groups, which may either form part of the main polymer chain or may be borne by a side substituent directly attached to the main polymer chain.

The cationic polymers generally have a number-average molecular mass ranging from 500 to 5×10⁶ and, for example, ranging from 10³ to 3×10⁶.

Among the cationic polymers which may be mentioned, for example, are polymers of polyamine, polymers of polyamino amide and polymers of polyquaternary ammonium. These polymers are known in the art.

The polymers of polyamine, polymers of polyamino amide, and polymers of polyquaternary ammonium are described, for example, in French Patents Nos 2 505 348 and 2 542 997. Among the said polymers, mention may be made of:

(1) Homopolymers or copolymers derived from acrylic or methacrylic esters or amides and comprising at least one unit chosen from units of formula (42), (43), (44) and (45) below:

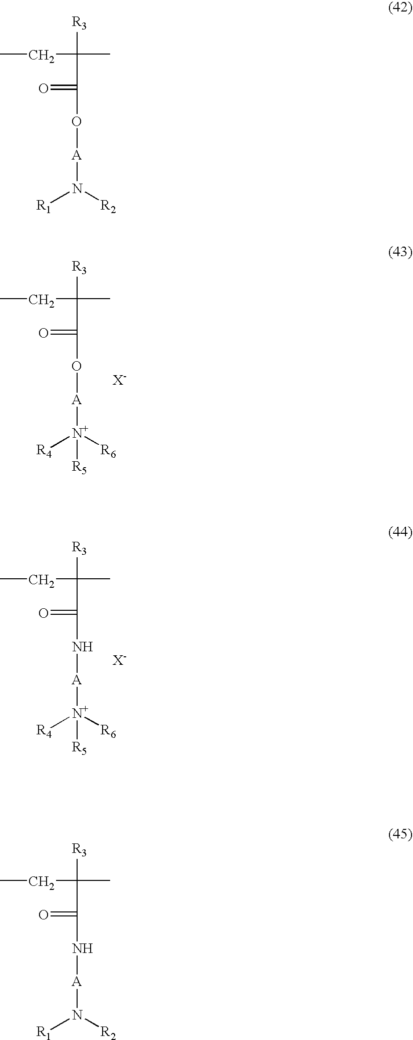

wherein:

R₃ is chosen from hydrogen and CH₃ radicals;

A is chosen from linear or branched alkyl groups of 1 to 6 carbon atoms, such as 2 or 3 carbon atoms, and hydroxyalkyl groups of 1 to 4 carbon atoms;

R₄, R₅ and R₆, which may be identical or different, are chosen from alkyl groups comprising from 1 to 6 carbon atoms, for example, alkyl groups comprising from 1 to 6 carbon atoms, and a benzyl radical;

R₁ and R₂, which may be identical or different, are chosen from hydrogen and alkyl groups comprising from 1 to 6 carbon atoms, such as methyl and ethyl groups;

X⁻ is an anion chosen from anions derived from inorganic and organic acids, such as a methosulphate anion and halides, such as chloride and bromide.

The polymers of family (1) can also comprise at least one unit derived from comonomers, that may be chosen from acrylamides, methacrylamides, diacetone acrylamides, acrylamides and methacrylamides substituted on the nitrogen with at least one group chosen from lower (C₁-C₄) alkyl groups, acrylic acids, methacrylic acids, acrylic esters, methacrylic esters, vinyllactam groups, such as vinylpyrrolidone and vinylcaprolactam, and vinyl esters.

Thus, the polymers of family (1), may be chosen, for example, from:

copolymers of acrylamide and of dimethylaminoethyl methacrylate quaternized with dimethyl sulphate or with a dimethyl halide, such as the product sold under the name Hercofloc by the company Hercules,

copolymers of acrylamide and of methacryloyloxyethyltrimethylammonium chloride disclosed, for example, in Patent Application No. EP-A-080 976 and sold under the name Bina Quat P 100 by the company Ciba Geigy,

copolymers of acrylamide and of methacryloyloxy-ethyltrimethylammonium methosulphate sold under the name Reten by the company Hercules,

quaternized and non-quaternized vinylpyrrolidone/dialkylaminoalkyl acrylate and methacrylate copolymers, such as the products sold under the name “Gafquat” by the company ISP, such as, for example, “Gafquat 734” or “Gafquat 755”, or the products known as “Copolymer 845, 958 and 937”. These polymers are described in detail in French Patents Nos. 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyidimethylamine copolymers sold, for instance, under the name Styleze CC 10 by ISP, and

quaternized vinylpyrrolidone/dimethylaminopropyl methacrylamide copolymers, such as the product sold under the name “Gafquat HS 100” by the company ISP.

(2) The cellulose ether derivatives comprising quaternary ammonium groups, disclosed in French Patent No. 1 492 597, and, for example, polymers sold under the name “JR” (JR 400, JR 125 and JR 30M) or “LR” (LR 400 or LR 30M) by the company Union Carbide Corporation. These polymers are also defined in the CTFA dictionary as quaternary ammoniums of hydroxyethylcellulose that have reacted with an epoxide substituted with a trimethylammonium group.

(3) cationic cellulose derivatives, such as cellulose copolymers and cellulose derivatives grafted with a water-soluble quaternary ammonium monomer, and described, for example, in U.S. Pat. No. 4,131,576, such as hydroxyalkylcelluloses, for instance hydroxymethyl-, hydroxyethyl- and hydroxypropylcelluloses grafted, for example, with a salt chosen from methacryloylethyltrimethylammonium, methacrylamidopropyltrimethylammonium and dimethyldiallylammonium salts.

The commercial products corresponding to this definition are, for example, the products sold under the names “Celquat L 200” and “Celquat 100 H” by the company National Starch.

(4) The cationic polysaccharides described more, for instance, in U.S. Pat. Nos. 3,589,578 and 4,031,307, such as guar gums comprising cationic trialkylammonium groups. For example, guar gums modified with a salt (e.g. chloride) of 2,3-epoxypropyltrimethylammonium are used.

Such products are sold, for example, under the trade names Jaguar C13 S, Jaguar C 15, Jaguar C 17, and Jaguar C162 by the company Meyhall.

(5) Polymers comprising piperazinyl units and divalent alkylene and hydroxyalkylene radicals comprising straight and branched chains, optionally interrupted by at least one atom chosen from oxygen, sulphur and nitrogen atoms or by at least one aromatic or heterocyclic ring, as well as at least one of the oxidation and/or quaternization products of these polymers. Such polymers are described, for example, in French Patents Nos. 2 162 025 and 2 280 361.

(6) Water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French Patents Nos. 2 252 840 and 2 368 508.

(7) The polyamino amide derivatives resulting from the condensation of polyalkylene polyamines with polycarboxylic acids followed by alkylation with difunctional agents. Non-limiting mention may be made, for example, of adipic acid/dialkylaminohydroxyalkyldialkylenetriamine polymers wherein the alkyl radical comprises from 1 to 4 carbon atoms and, for example, may be chosen from methyl, ethyl and propyl groups. Such polymers are described, for instance, in French Patent No. 1 583 363.

Among these derivatives, mention may be made, for example, of the adipic acid/dimethylaminohydroxypropyl/diethylenetriamine polymers sold under the name “Cartaretine F, F4 or F8” by the company Sandoz.

(8) The polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 6 carbon atoms. The molar ratio between the polyalkylene polyamine and the dicarboxylic acid may range, for example, from 0.8:1 to 1.4:1; the polyamino amide resulting from the reaction may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Other non-limiting examples of such derivates include the adipic acid/epoxypropyl/diethylenetriamine copolymer sold, for example, under the name “Hercosett 57” by the company Hercules Inc. or under the name “PD 170” or “Delsette 101” by the company Hercules.

(9) Cyclopolymers of alkyldiallylamine or of dialkyldiallylammonium, such as the homopolymers or copolymers comprising, as main constituent of the chain, at least one unit corresponding to formula (46) or (47):

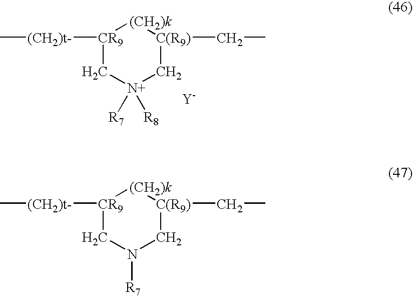

wherein

k and t are equal to 0 or 1, the sum k+t being equal to 1; R₉ is chosen from hydrogen and methyl radicals; R₇ and R₈, which may be identical or different, are chosen from alkyl groups comprising from 1 to 6 carbon atoms, hydroxyalkyl groups wherein the alkyl group, for example, comprises from 1 to 5 carbon atoms, lower (C₁-C₄) amidoalkyl groups, and R₇ and R₈ can form, together with the nitrogen atom to which they are attached, heterocyclic groups, such as piperidyl and morpholinyl; additionally, R₇ and R₈, which may be identical or different, are chosen from alkyl groups comprising from 1 to 4 carbon atoms; Y⁻ is an anion, such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

Among the polymers defined above, non-limiting mention may be made, for example, of the dimethyldiallylammonium chloride homopolymer sold under the name “Merquat 100” by the company Calgon (and its homologues of low weight-average molecular mass) and the copolymers of diallyidimethylammonium chloride and of acrylamide, sold under the name “Merquat 550.”

(10) The quaternary diammonium polymer comprising repeating units corresponding to the formula (48):

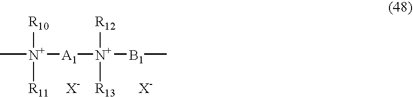

wherein):

R₁₀, R₁₁, R₁₂ and R₁₃, which may be identical or different, are chosen from liphatic, alicyclic and arylaliphatic radicals comprising from 1 to 6 carbon atoms and from lower hydroxyalkylaliphatic radicals, or R₁₀, R₁₁, R₁₂ and R₁₃, together or separately, form, together with the nitrogen atoms to which they are attached, heterocycles optionally comprising a second hetero atom other than nitrogen, or R₁₀, R₁₁, R₁₂ and R₁₃ are chosen from linear and branched C₁-C₆ alkyl radicals substituted with nitrile, ester, acyl and amide group and groups of formulae —CO—O—R₁₄-D and —CO—NH—R₁₄-D wherein R₁₄ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen and sulphur atoms and sulphoxide, sulphone, disulphide, amino, alkylamino, hydroxyl, quaternary ammonium, ureido, amide and ester groups, and

X⁻ is an anion chosen from anions derived from mineral and organic acids;

A₁, R₁₀ and R₁₂ may optionally form, with the two nitrogen atoms to which they are attached, a piperazine ring. In addition, if A₁ is a radical chosen from linear and branched, saturated and unsaturated alkylene and hydroxyalkylene radicals, B₁ can also be chosen from the group —(CH₂)ₙ—CO-D-OC—(CH₂)ₙ— wherein n is an integer ranging from 1 to 100, such as from 1 to 50.

D is chosen from:

a) glycol residues of formula: —O-Z-O—, wherein Z is chosen from linear and branched hydrocarbon-based radicals and groups corresponding to one of the following formulae:

—(CH₂—CH₂—O)ₓ—CH₂—CH₂—

—[CH₂—CH(CH₃)—O]_{y}—CH₂—CH(CH₃)—

wherein x and y, which may be identical or different, are each an integer chosen from 1 to 4, representing a defined and unique degree of polymerization or any number ranging from 1 to 4 representing an average degree of polymerization;

b) bis-secondary diamine residues, such as a piperazine derivative;

c) bis-primary diamine residues of formula: —NH—Y—NH—, wherein Y is chosen from linear and branched hydrocarbon-based radicals, and the divalent radical

—CH₂—CH₂—S—S—CH₂—CH₂—; and

d) ureylene groups of formula: —NH—CO—NH—.

In one aspect of the disclosure, X⁻ is an anion, such as chloride or bromide.

These polymers may have a number-average molecular mass ranging from 1000 to 100 000.

These polymers are described, for example, in French Patents Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, the polymers that comprise repeating units corresponding to formula (49) below can be used:

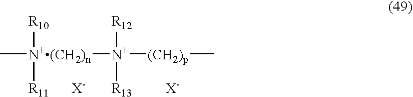

wherein R₁₀, R₁₁, R₁₂ and R₁₃, which may be identical or different, are chosen from alkyl and hydroxyalkyl radicals comprising from 1 to 4 carbon atoms, n and p, which may be identical or different, are integers ranging from 2 to 20, and X⁻ is an anion chosen from anions derived from mineral and organic acids.

(11) polyquaternary ammonium polymers comprising of units of formula (50):

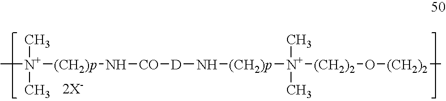

wherein:

p is an integer ranging from 1 to 6, D may be nothing or may be chosen from groups of formula —(CH₂)ᵣ—CO— wherein r is an integer chosen from 4 to 7, and

X⁻ is an anion chosen from anions derived from mineral and organic acids.

The cationic polymers comprising units of formula (50) are described, for example, in Patent Application No. EP-A-122 324 and may be prepared according to the processes described in U.S. Pat. Nos. 4,157,388, 4,390,689, 4,702,906 and 4,719,282.

Among these polymers, non-limiting mention may be made of those with a molecular mass measured by carbon-13 NMR of less than 100,000, for which p is equal to 3, and

a) D is chosen from —(CH₂)₄—CO— groups, X is chosen from chlorine, and the molecular mass measured by carbon-13 NMR (¹³C NMR) is generally 5,600. A polymer of this type is sold by the company Miranol under the name Mirapol-AD1,

b) D is chosen from —(CH₂)₇—CO— groups, X is chosen from chlorine, and the molecular mass measured by carbon-13 NMR (¹³C NMR) is generally 8100. A polymer of this type is sold by the company Miranol under the name Mirapol-AZ1,

c) D is equal to zero, X is chosen from chlorine, and the molecular mass measured by carbon-13 NMR (¹³C NMR) is generally 25,500. A polymer of this type is sold by the company Miranol under the name Mirapol-A15,

d) “block copolymers” formed from units corresponding to the polymers described in paragraphs a) and c), sold by the company Miranol under the names Mirapol-9 (¹³C NMR molecular mass, about 7800), Mirapol-175 (¹³C NMR molecular mass, about 8000) and Mirapol-95 (¹³C NMR molecular mass, about 12 500).

In one aspect of the disclosure, the polymer comprises units of formula (50) wherein p is equal to 3, D is equal to zero, X is chosen from chlorine, and the molecular mass measured by carbon-13 NMR (¹³C NMR) is generally 25,500.

(12) Quaternary polymers of vinylpyrrolidone and of vinylimidazole, such as the products sold under the names Luviquat FC 905, FC 550 and FC 370 by the company BASF.

(13) Polyamines such as Polyquart H sold by Henkel, under the reference name “Polyethylene glycol (15) tallow polyamine” in the CTFA dictionary.

(14) Crosslinked methacryloyloxy(C₁-C₄)alkyltri(C₁-C₄)alkylammonium salt polymers, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide. A crosslinked acrylamide/methacryloyloxyethyltrimethylammonium chloride copolymer (20/80 by weight) in the form of a dispersion comprising 50% by weight of the said copolymer in mineral oil can, for example, be used. This dispersion is sold under the name “Salcare® SC 92” by the company Allied Colloids. In another aspect of the disclosure, a crosslinked methacryloyloxyethyltrimethylammonium chloride homopolymer comprising about 50% by weight of the homopolymer in mineral oil or in a liquid ester can also be used. These dispersions are sold under the names “Salcare® SC 95” and “Salcare® SC 96” by the company Allied Colloids.

Other cationic polymers that can be used in the context of the disclosure are polyalkyleneimines, such as polyethyleneimines, polymers comprising vinylpyridine and vinylpyridinium units, condensates of polyamines and of epichlorohydrin, quaternary polyureylenes and chitin derivatives.

Among all the cationic polymers that may be used, non-limiting examples include the polymers of families (1), (9), (10), (11) and (14) and, in another aspect of the disclosure, the polymers comprising repeating units of formulae (W) and (U) below can be used:

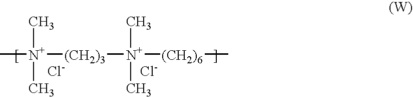

and, for example, those polymers comprising repeating units of formula (W) whose number-average molecular weight, determined by gel permeation chromatography, ranges from 9500 to 9900;

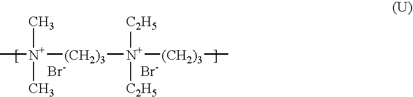

and, for example, those polymers comprising repeating units of formula (U) whose number-average molecular weight, determined by gel permeation chromatography, is about 1200.

Amphoteric Polymers

The amphoteric polymers, disclosed herein, may be chosen from polymers comprising units K and M randomly distributed in the polymer chain, wherein K is a unit derived from a monomer comprising at least one basic nitrogen atom and M is a unit derived from an acidic monomer comprising at least one carboxylic and sulphonic groups, or K and M, which may be identical or different, may be chosen from groups derived from zwitterionic monomers of carboxybetaine or of sulphobetaine;

K and M, which may be identical or different, may also be chosen from cationic polymer chains comprising at least one group chosen from primary, secondary, tertiary and quaternary amine groups, wherein at least one of the amine groups comprises carboxylic or sulphonic groups linked via a hydrocarbon-based radical, or K and M can form part of a chain of a polymer comprising an α,β-dicarboxylic ethylene unit wherein one of the carboxylic groups has been made to react with a polyamine comprising at least one group chosen from primary and secondary amine groups.

The amphoteric polymers corresponding to the above definition, for example, are chosen from the following polymers:

(1) Polymers resulting from the copolymerization of at least one monomer derived from a vinyl compound bearing a carboxylic group such as acrylic acid, methacrylic acid, maleic acid, α-chloroacrylic acid, and at least one monomer derived from a substituted vinyl compound comprising at least one basic atom, such as dialkylaminoalkyl methacrylate and acrylate, dialkylaminoalkylmethacrylamide and -acrylamide. Such compounds are described in U.S. Pat. No. 3,836,537.

Mention may also be made of the sodium acrylate/acrylamidopropyltrimethylammonium chloride copolymer sold under the name Polyquart KE 3033 by the company Henkel.

The substituted vinyl compound comprising at least one basic atom may also be chosen from dialkyldiallylammonium salts, such as dimethyidiallylammonium chloride.

The copolymers of acrylic acid and of the latter monomer are sold under the names Merquat 280, Merquat 295 and Merquat Plus 3330 by the company Calgon.

(2) Polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer, such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one aspect of the disclosure, the N-substituted acrylamides or methacrylamides are, for example, groups wherein the alkyl radicals comprise from 2 to 6 carbon atoms, such as N-ethylacrylamide, N-tert-butylacrylamide, and the corresponding methacrylamides.

The acidic comonomers are chosen, for example, from acrylic acids, methacrylic acids, crotonic acids, itaconic acids, maleic acids and fumaric acids and alkyl monoesters, comprising from 1 to 4 carbon atoms, of maleic or fumaric acids or anhydrides.

The basic comonomers are chosen, for example, from aminoethyl, butylaminoethyl, N,N′-dimethylaminoethyl, and N-tert-butylaminoethyl methacrylates.

The copolymers having the CTFA (4th edition, 1991) name octylacrylamide/acrylates/butylaminoethyl methacrylate copolymer such as the products sold under the name Amphomer or Lovocryl 47 by the company National Starch are, for example, used.

(3) Crosslinked and alkylated partially or totally polyamino amides derived from polyamino amides of general formula:

—[—CO—R₁₉—CO—Z—]— (51)

wherein R₁₉ is chosen from divalent radicals derived from saturated dicarboxylic acids, mono- or dicarboxylic aliphatic acids comprising ethylenic double bonds, esters of a lower alkanol, comprising from 1 to 6 carbon atoms, of these acids and a radical derived from the addition of any one of the acids to a bis(primary) or bis(secondary) amine, and Z is a radical chosen from bis(primary), mono- or bis(secondary) polyalkylene-polyamine radicals and, for example, Z represents:

a) in proportions ranging from 60 to 100 mol %, the radical of formula (52)

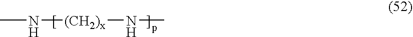

wherein x=2 and p=2 or 3, or x=3 and p=2

this radical being derived from a compound chosen from diethylenetriamine, triethylenetetraamine and dipropylenetriamine;

b) in proportions ranging from 0 to 40 mol %, the radical (52) above wherein x =2 and p =1 and which is derived from a compound chosen from ethylenediamine and piperazine:

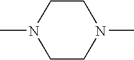

c) in proportions ranging from 0 to 20 mol %, the —NH—(CH₂)₆—NH— radical, which is derived from hexamethylenediamine, these polyamino amines can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one aspect of the disclosure, the saturated carboxylic acids are chosen from acids comprising from 6 to 10 carbon atoms, such as adipic acid, 2,2,4-trimethyladipic acid and 2,4,4-trimethyladipic acid, terephthalic acid and acids comprising ethylenic double bonds, such as acrylic acid, methacrylic acid, and itaconic acid.

The alkane sultones used in the alkylation are chosen, for example, from propane sultone and butane sultone, and salts of the alkylating agents are chosen, for example, from sodium and potassium salts.

(4) Polymers comprising zwitterionic units of formula (53):

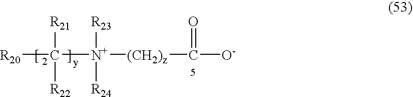

wherein R₂₀ is chosen from polymerizable unsaturated groups, such as acrylate, methacrylate, acrylamide and methacrylamide groups, y and z, which may be identical or different, are chosen from integers ranging from 1 to 3, R₂₁, and R₂₂, which may be identical or different, are chosen from hydrogen, methyl, ethyl and propyl groups, R₂₃ and R₂₄, which may be identical or different, are chosen from hydrogen and alkyl radicals, such that the sum of the carbon atoms in R₂₃ and R₂₄ does not exceed 10.

The polymers comprising such units can also comprise units derived from nonzwitterionic monomers, such as monomers chosen from dimethyl and diethylaminoethyl acrylates, dimethyl and diethylaminoethyl methacrylates, alkyl acrylates, alkyl methacrylates, acrylamides, methacrylamides and vinyl acetate.

By way of example, mention may be made of the copolymer of butyl methacrylate/dimethylcarboxymethylammonioethyl methacrylate, such as the product sold under the name Diaformer Z301 by the company Sandoz.

(5) Polymers derived from chitosan, described, for example, in French Patent No. 2 137 684 or U.S. Pat. No. 3,879,376, comprising monomer units corresponding to formulae (54), (55) and (56) below combined in their chain:

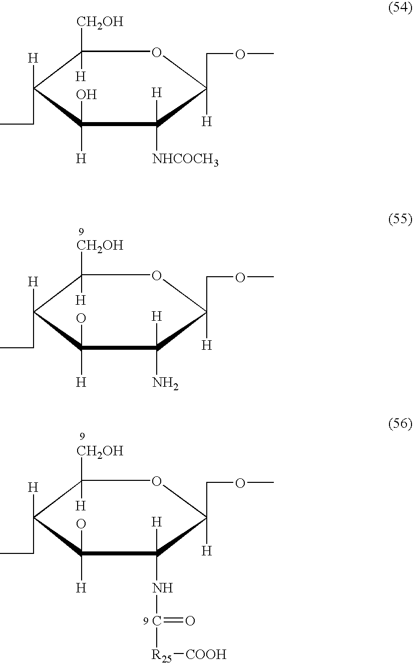

the unit (54) being present in proportions ranging from 0 to 30%, the unit (55) in proportions ranging from 5% to 50% and the unit (56) in proportions ranging from 30% to 90%, and wherein in unit (56), R₂₅ is a radical of formula:

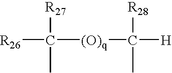

wherein q is an integer chosen from zero and 1;

if q=0, R₂₆, R₂₇ and R₂₈, which may be identical or different, are each chosen from hydrogen, methyl, hydroxyl, acetoxy and amino residues, monoalkylamine residues and a dialkylamine residues which are optionally interrupted by at least one nitrogen and optionally substituted with at least one amine, hydroxyl, carboxyl, alkylthio and sulphonic groups, and alkylthio residues wherein the alkyl group comprises amino residues, at least one of the radicals R₂₆, R₂₇ and R₂₈ being, in this case, a hydrogen atom;

or, if q=1, R₂₆, R₂₇ and R₂₈, which may be identical or different, areeach chosen from hydrogen and salts formed by these compounds with bases or acids.

The polymers comprising of unit (54) are present in an amount ranging, for example, from 0 to 20% by weight, relative to the total weight of the polymer, the polymers comprising of unit (55) are present, for example, in an amount ranging from 40% to 50% by weight, relative to the total weight of the polymer and polymers comprising of unit 56 are present, for instance, in an amount ranging from 40% to 50% by weight, relative to the total weight of the polymer and wherein R₂₅ is chosen from radical —CH₂—CH₂—.

(6) Polymers derived from the N-carboxyalkylation of chitosan, such as N-carboxymethylchitosan or N-carboxybutylchitosan sold under the name “Evalsan” by the company Jan Dekker.

(7) Polymers corresponding to the general formula (57) as described, for example, in French Patent No. 1 400 366:

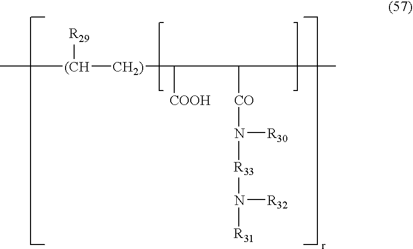

wherein R₂₉ is chosen from hydrogen, CH₃O, CH₃CH₂O and phenyl radicals, R₃₀ is chosen from hydrogen and lower alkyl radicals, such as methyl and ethyl groups, R₃₁ is chosen from hydrogen and lower alkyl radicals, such as methyl and ethyl radicals, R₃₂ is chosen from lower alkyl radicals, such as methyl and ethyl radicals and radicals corresponding to the formula: —R₃₃—N(R₃₁)₂, wherein R₃₃ is chosen from —CH₂—CH₂—, —CH₂—CH₂—CH₂— or —CH₂—CH(CH₃)— groups, and R₃₁ is chosen from hydrogen and lower alkyl radicals, such as methyl and ethyl radicals,

and also the higher homologues of these radicals comprising up to 6 carbon atoms,

r is chosen such that the number-average molecular weight of said polymer ranges from 500 to 6 000 000, such as from 1000 to 1 000 000.

(8) Amphoteric polymers of the type -D-X-D-X- chosen from:

a) polymers obtained by the action of chloroacetic acid or sodium chloroacetate on compounds comprising at least one unit of formula:

-D-X-D-X-D- (58)

wherein D is chosen from a radical

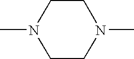

and X is chosen from the symbol E and E′, wherein E and E′, which may be identical or different, are chosen from divalent alkylene radicals comprising at least one chain chosen from straight and branched chains comprising up to 7 carbon atoms in the main chain, which may be optionally substituted with at least one hydroxyl group and which can further comprise at least one group chosen from oxygen, nitrogen and sulphur, and 1 to 3 aromatic and heterocyclic rings; wherein the oxygen, nitrogen and sulphur atoms can be present in the form of a group chosen from ether, thioether, sulphoxide, sulphone, sulphonium, alkylamine and alkenylamine groups, hydroxyl, benzylamine, amine oxide, quaternary ammonium, amide, imide, alcohol, ester and urethane groups;

b) polymers of formula:

-D-X-D-X- (59)

wherein D is chosen from a radical

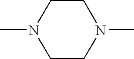

and X is chosen from the symbol E and E′ and at least once is chosen from E′; wherein E has the meaning defined above and E′ is chosen from straight and branched chain alkylene divalent radicals comprising up to 7 carbon atoms in the main chain, which may be optionally substituted with at least one hydroxyl radical and comprising at least one nitrogen atom, wherein the nitrogen atom is substituted with an alkyl chain wherein that optionally comprises an oxygen atom and further the straight-chain and branched-chain alkylene divalent groups, comprising up to 7 carbon atoms in the main chain can comprise at least one functional group chosen from carboxyl and hydroxyl groups and wherein the alkyl chain can be betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine, such as N,N-dimethylaminopropylamine or by semiesterification with an N,N-dialkanolamine. These copolymers can also comprise other vinyl comonomers, such as vinylcaprolactam.

Among all the cationic or amphoteric polymers that may be used non-limiting mention may be made of:

(i) among the cationic polymers:

dimethyidiallylammonium chloride homopolymers sold under the name Merquat 100DRY by the company Merck;

copolymers of dimethyldiallylammonium chloride and of acrylamide that are sold under the name Merquat 2200 by the company Calgon;

polymers of poly(quaternary ammonium) type prepared and described in French Patent No. 2 270 846, comprising of repeating chosen from units of formulae (W) and (U) below are used:

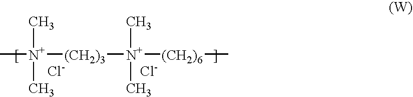

and, for example, those whose number-average molecular weight, determined by gel permeation chromatography, ranges from 9500 to 9900;

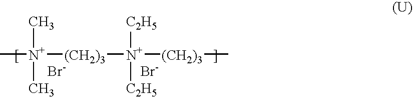

and, for example, those whose number-average molecular weight, determined by gel permeation chromatography, is about 1200;

polymers of poly(quaternary ammonium) type of family (11) and of formula (50) wherein X⁻ is chosen from chlorine, such as those whose weight-average molecular mass is less than 100 000 and, for example, less than or equal to 50 000;

(ii) among the amphoteric polymers:

dimethyldiallylammonium chloride/acrylic acid (80/20) copolymers sold under the name Merquat 280 Dry by the company Calgon (CTFA name: Polyquaternium 22);

dimethyldiallylammonium chloride/acrylic acid (95/5) copolymers sold under the name Merquat 295 Dry by the company Calgon (CTFA name: Polyquaternium 22);

copolymers of methacrylamidopropyltrimonium chloride, of acrylic acid and of ethyl acrylate, sold under the name Merquat 2001 by the company Calgon (CTFA name: Polyquaternium 47); and

acrylamide/dimethyldiallylammonium chloride/acrylic acid terpolymers sold under the name Merquat Plus 3330 Dry by the company Calgon (CTFA name: Polyquaternium 39).

When the polymers are present in the simultaneous anhydrous bleaching and dyeing pastes, as disclosed herein, the cationic and/or amphoteric polymers are present in a weight proportion of less than or equal to 20% relative to the total weight of the said paste and, for instance, less than or equal to 8%.

Moreover, it is a common practice to thicken or gel bleaching compositions with conventional thickeners, such as water-soluble thickening polymers, for instance cellulose derivatives, starch derivatives, crosslinked polyacrylic acid, alginates and thickening silicas in order to localize the bleaching and dyeing product in application to the hair, so that it does not run down the face or outside the areas that are proposed to be treated,.

According to the disclosure, the pasty anhydrous compositions for simultaneous bleaching and dyeing, may also comprise at least one gelling agent chosen fumed silicas of hydrophilic or hydrophobic nature and at least one block polymer comprising at least one unit chosen from alkylene and alkylene oxide units.

Among the fumed silicas of hydrophilic nature that may, for example, be used non-limiting mention may be made of those sold by the company Degussa Hüls under the trade names Aerosils® 90, 130, 150, 200, 300 and 380.

Also, among the fumed silicas of hydrophobic nature that may, for instance, be used non-limiting mention may be made of those sold by the company Degussa Hüls under the trade name Aerosil® R202, R805, R812, R972 and R974.

Additionally, among the block polymers comprising at least one unit chosen from alkylene and alkylene oxide units, non-limiting mention may, for example, be made of diblock, triblock, multiblock and radial-block copolymers comprising of segments chosen from styrene monomers and thermoplastic monomers and comonomers, described in U.S. Pat. No. 5,221,534.

In one aspect of the disclosure, the block copolymers that can be used are those, for example, for which the at least one thermoplastic monomer or comonomer is chosen from C₃-C₄ ethylene/alkylenes, such as hydrogenated copolymers comprising styrene blocks and C₃-C₄ ethylene/alkylene blocks.

According to the present disclosure, a mixture of hydrogenated copolymers comprising butylene/ethylene and styrene blocks and of hydrogenated copolymer comprising ethylene/propylene and styrene blocks, in mineral oil, and, for example, a mixture of 1% to 20% by weight of hydrogenated copolymer comprising butylene/ethylene and styrene blocks and of hydrogenated copolymer comprising ethylene/propylene and styrene blocks in and wherein the mineral oil is in amount, for example, ranging from 80% to 99% by weight, relative to the weight of the composition, can be advantageously used.

Such mixtures are sold, for example, by the company Penreco under the trade names Versagel® M200 and Geahlene® 200 and Versagel® M750 and Geahlene® 750, or by the company Aiglon under the trade names Transgel® or Syngel® (90% liquid paraffin, 5% hydrogenated butylene/ethylene/styrene copolymer, 5% hydrogenated ethylene/propylene/styrene copolymer).

It is also possible to use the styrene/ethylene-butylene/styrene triblock polymers (INCI name “Hydrogenated styrene/butadiene copolymer”) sold by the company Shell Chimie under the trade names Kraton® G-1650, G-1652 and G-1657.

As disclosed herein, the pasty compositions comprise at least one gelling agent present in an amount, for example, ranging from 0.01% to 10% by weight, relative to the total weight of the composition, for example, from 0.01% to 5% by weight, relative to the total weight of the composition and, further for example, in an amount ranging from 0.1% to 2.5% by weight, relative to the total weight of the composition.

The pasty anhydrous compositions for simultaneous bleaching and dyeing, as disclosed, may also comprise at least one water-soluble thickening polymer.

Water-soluble Thickening Polymers

According to the present disclosure, the water-soluble thickening polymers comprise any water-soluble polymer that is synthetic or of natural origin, conventionally used in the cosmetic field, and other than the nonionic and/or anionic amphiphilic polymers comprise at least one fatty chain, as disclosed herein.

As examples of synthetic polymers, non-limiting mention may be made of polyvinylpyrrolidone, polyacrylic acids, polyacrylamide, non-crosslinked poly-2-acrylamidopropanesulphonic acids, such as the product sold under the name Simugel EG by the company SEPPIC, crosslinked poly-2-acrylamido-2-methylpropanesulphonic acid, poly-2-acrylamido-2-methylpropanesulphonic acid crosslinked and partially neutralized with aqueous ammonia sold under the brand name Hostacerin AMPS by the company Clariant, mixtures with a synergistic thickening effect of the non-crosslinked poly-2-acrylamido-2-methylpropanesulphonic acid with hydroxyalkylcellulose ethers or with poly(ethylene oxide) as described in U.S. Pat. No. 4,540,510, or mixtures with a synergistic thickening effect of a poly(meth)acrylamido(C₁-C₄)alkylsulphonic acid preferably crosslinked with a crosslinked copolymer of maleic anhydride and of a (C₁-C₅)alkyl vinyl ether, such as the mixture Hostacerin AMPS/Stabileze QM (from the company ISF) and as described in the Inventor's French Patent Application No. 0 014 416.

As disclosed herein, non-limiting examples of the thickening polymers of natural origin that may be used include polymers comprising at least one sugar unit, such as nonionic guar gums; biopolysaccharide gums of microbial origin, such as scieroglucan gum and xanthan gum; gums derived from plant exudates, such as gum arabic, ghatti gum, karaya gum, gum tragacanth, carrageenan gum, agar gum and carob gum; pectins; alginates; starches; hydroxy(C₁-C₆)alkylcelluloses and carboxy(C₁-C₆)alkylcelluloses.

For the purposes of the present disclosure, the expression “sugar unit” means a monosaccharide portion (i.e. monosaccharide or oside or simple sugar) or an oligosaccharide portion (for instance, short chains formed from the linking of monosaccharide units, which may be identical or different) or a polysaccharide portion [such as long chains comprising monosaccharide units, which may be identical or different, i.e. polyholosides and polyosides (for example, homopolyosides and heteropolyosides)]. The saccharide units can also be substituted with alkyl, hydroxyalkyl, alkoxy, acyloxy or carboxyl radicals, and alkyl radicals comprising from 1 to 4 carbon atoms.

The nonionic guar gums can optionally be modified.

The unmodified guar gums are, for example, the products sold under the name Guargel D/15 by the company Goodrich, Vidogum GH 175 by the company Unipectine and under the names Meypro-Guar 50 and Jaguar C by the company Meyhall.

The modified nonionic guar gums are, for instance, modified with C₁-C₆ hydroxyalkyl groups.

Among the hydroxyalkyl groups that may be mentioned, for example, are hydroxymethyl, hydroxyethyl, hydroxypropyl and hydroxybutyl groups.

These guar gums are known in the prior art and can be prepared, for example, by reacting the corresponding alkene oxides, such as propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum ranges, for example, from 0.4 to 1.2.

Such nonionic guar gums optionally modified with hydroxyalkyl groups are sold, for example, under the trade names Jaguar HP8, Jaguar HP60 and Jaguar HP120, Jaguar DC 293 and Jaguar HP 105 by the company Rhone-Poulenc (Meyhall) or under the name Galactasol 4H4FD2 by the company Aqualon.

The biopolysaccharide gums of microbial origin, such as scleroglucan and xanthan gums, gums derived from plant exudates such as arabic, ghatti, karaya, tragacanth, carrageenan, agar and carob gums, hydroxyalkylcelluloses and carboxymethylcelluloses, pectins, alginates and starches are known to those skilled in the art and are described, for example, in the book by Robert L. Davidson entitled “Handbook of Water soluble gums and resins” published by McGraw Hill Book Company (1980).

Among these gums, the scleroglucans are chosen from products sold under the name Actigum CS by the company Sanofi Bio Industries, such as Actigum CS 11, and under the name Amigel by the company Alban Muller International. Other scleroglucans, such as the one treated with glyoxal in French Patent Application No. 2 633 940, can also be used.

The xanthans are chosen from products sold under the names Keltrol, Keltrol T, Keltrof TF, Keltrol BT, Keltrol RD and Keltrol CG by the company Nutrasweet Kelco, or under the names Rhodicare S and Rhodicare H by the company Rhodia Chimie.

Among the starch derivatives that may be mentioned, for example, is the product sold under the name Primogel by the company Avebe.

The hydroxy(C₁-C₆)alkylcelluloses are chosen from hydroxyethylcelluloses, such as those sold under the names Cellosize QP3L, Cellosize QP4400H, Cellosize QP30000H, Cellosize HEC30000A and Cellosize Polymer PCG10 by the company Amerchol, or Natrosol 250HHR, Natrosol 250MR, Natrosol 250M, Natrosol 250HHXR, Natrosol 250HHX, Natrosol 250HR and Natrosol HX by the company Hercules, or Tylose H1000 by the company Hoechst.

The hydroxy(C₁-C₆)alkylcelluloses are also chosen, for example, from hydroxypropylcelluloses, such as the products sold under the names Klucel EF, Klucel H, Klucel LHF, Klucel MF and Klucel G by the company Aqualon.

Non-limiting examples of the carboxy(C₁-C₆)alkylcelluloses used include carboxymethylcelluloses, for which mention may be made of the products sold under the names Blanose 7M8/SF, Blanose Raffinée 7M, Blanose 7LF, Blanose 7MF, Blanose 9M31F, Blanose 12M31XP, Blanose 12M31P, Blanose 9M31XF, Blanose 7H, Blanose 7M31 and Blanose 7H3SXF by the company Aqualon, or Aquasorb A500 and Ambergum 1221 by the company Hercules, or Cellogen HP810A and Cellogen HP6HS9 by the company Montello, or Primellose by the company Avebe.

When the water-soluble thickening polymers are present in the pasty anhydrous compositions disclosed herein, they are present in an amount ranging from 0.01% to 30% by weight, relative to the total weight of the composition and, for example, ranging from 0.01% to 15% by weight, relative to the total weight of the composition.

Other Adjuvants

As disclosed herein, the pasty anhydrous composition for simultaneous bleaching and dyeing may also comprise at least one wax chosen from hydrocarbon-based waxes, fluoro waxes and silicone waxes, and mixtures thereof. The silicone waxes may be chosen from waxes comprising silicone structures and units comprising at least one linear and branched alkyl and alkoxy chain comprising from 10 to 45 carbon atoms that can be pendant and at the end of a silicone structure. For example, these waxes are known, respectively, as alkyl dimethicones and alkoxy dimethicones. Moreover, these alkyl chains may comprise at least one ester function. As other waxes that may be used in the invention, non-limiting mention may be made of waxes of animal origin, for instance lanolin and beeswax; waxes of plant origin, for instance carnauba wax and candelilla wax; waxes of mineral origin, for example paraffin wax, lignite wax and microcrystalline waxes, ceresin and ozokerite; synthetic waxes, for instance polyethylene waxes; and mixtures thereof.

For example, the compositions, as disclosed herein, may comprise beeswax.

The pasty anhydrous composition according to the disclosure may also comprise at least one filler, such as clays, amorphous silica, binders, such as vinylpyrrolidone, lubricants, for instance polyol stearates and alkali metal and alkaline-earth metal stearates, and also agents used for controlling the evolution of oxygen, such as magnesium carbonates and magnesium oxides, coloring agents and matting agents, for instance titanium oxides, or anionic, nonionic, cationic or amphoteric surfactants.

For instance, the compositions of the invention can comprise at least one surfactant.

The surfactants may be chosen, alone or as mixtures, from anionic, amphoteric, nonionic, zwitterionic and cationic surfactants. According the present disclosure, non-limiting mention may be made of the following surfactants used:

(i) Anionic Surfactant(s):

Non-limiting examples of anionic surfactants that can be used, in the context of the present disclosure, include: salts (such as alkali metal salts, for example, sodium salts, ammonium salts, amine salts, amino alcohol salts and magnesium salts) and compounds, such as alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; (C₆-C₂₄)alkyl sulphosuccinates, (C₆-C₂₄)alkyl ether sulphosuccinates, (C₆-C₂₄)alkylamide sulphosuccinates; (C₆-C₂₄)alkyl sulphoacetates; (C₆-C₂₄)acyl sarcosinates and (C₆-C₂₄)acyl glutamates. It is also possible to use (C₆-C₂₄)alkylpolyglycoside carboxylic esters, such as alkylglucoside citrates, alkylpolyglycoside tartrates and alkylpolyglycoside sulphosuccinates, alkylsulphosuccinamates; acyl isethionates and N-acyl taurates, alkyl and acyl radicals of all of these different compounds, for example, comprising from 12 to 20 carbon atoms and wherein the aryl radical is chosen, for example from phenyl and benzyl groups. Among the anionic surfactants which can also be used, non-limiting mention may be made of fatty acid salts, such as oleic, ricinoleic, palmitic and stearic acid salts, copra oil acids and hydrogenated copra oil acids; acyl lactylates wherein the acyl radical comprises 8 to 20 carbon atoms. Also, It is possible to use alkyl D-galactoside uronic acids and their salts, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, such as those comprising from 2 to 50 alkylene oxide groups, for example, ethylene oxide groups, and mixtures thereof.

(ii) Nonionic Surfactant(s):

The nonionic surfactants are also compounds that are well known in the prior art (see, for example, “Handbook of Surfactants” by M. R. Porter, published by Blackie & Son (Glasgow and London), 1991, pp. 116-178). Thus, the nonionic surfactants can be chosen, for example, from polyethoxylated and polypropoxylated alkylphenols, alpha-diols and alcohols, comprising fatty chains comprising, for example, from 8 to 18 carbon atoms, the number of ethylene oxide or propylene oxide groups can range, for example, from 2 to 50. non-limiting mention may also be made of copolymers of ethylene oxide and propylene oxide, condensates of ethylene oxide and propylene oxide with fatty alcohols; polyethoxylated fatty amides comprising, for example, from 2 to 30 mol of ethylene oxide, polyglycerolated fatty amides comprising, for example, from 1 to 5, such as from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, and amine oxides, such as (C₁₀-C₁₄)alkylamine oxides and N-acylaminopropyl-morpholine oxides. In the context of the present disclosure, the alkylpolyglycosides can be chosen from nonionic surfactants.

(iii) Amphoteric or Zwitterionic Surfactant(s):

The amphoteric or zwitterionic surfactants can be chosen, for example, from aliphatic secondary and tertiary amine derivatives wherein the linear and branched chain aliphatic radical comprises from 8 to 18 carbon atoms and comprises at least one water-solubilizing anionic group (for example, carboxylate, sulphonate, sulphate, phosphate and phosphonate groups); non-limiting mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

Among the amine derivatives, non-limiting mention may be made of the products sold under the name Miranol, as described in U.S. Pat. Nos. 2,528,378 and 2,781,354 and classified in the CTFA dictionary, 3rd edition, 1982, under the names Amphocarboxyglycinates and Amphocarboxypropionates chosen from the formula below:

R₂—CONHCH₂CH₂—N(R₃)(R₄)(CH₂COO⁻)

wherein: R₂ is chosen from linear and branched C₅-C₂₀ alkyl radicals derived, for example, from R₂—COOH acids present in hydrolyzed copra oil, heptyl, nonyl and undecyl radicals, R₃ is chosen from beta-hydroxyethyl groups and R₄ is chosen from carboxymethyl groups;

and the formula below:

R₂′—CONHCH₂CH₂—N(B)(C)

wherein:

B is chosen from —CH₂CH₂OX′, C is chosen from —(CH₂)_{z}—Y′, wherein z is an integer chosen from 1 and 2,

X′ is chosen from —CH₂CH₂—COOH groups and hydrogen,

Y′ is a radical chosen from —COOH and —CH₂—CHOH—SO₃H radicals,

R₂′ is chosen from saturated and unsaturated, linear and branched C₅-C₂₀ alkyl radicals chosen from R′₂-COOH acids present, for example, in copra oil and in hydrolyzed linseed oil, alkyl radicals, such as C₇, C₉, C₁₁ and C₁₃ alkyl radicals, C₁₇ alkyl radicals and its iso form, and unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid and Cocoamphodipropionic acid.

For example, non-limiting mention may be made of cocoamphodiacetate sold under the trade name Miranol® C2M concentrate by the company Rhodia Chimie.

(iv) Cationic Surfactants:

Among the cationic surfactants, non-limiting mention may be made, for example, of: salts of optionally polyoxyalkylenated primary, secondary and tertiary fatty amines; quaternary ammonium salts, such as tetraalkylammonium, alkylamidoalkyltrialkylammonium, trialkylbenzylammonium, trialkylhydroxyalkylammonium and alkylpyridinium chlorides and bromides; imidazoline derivatives; and amine oxides of cationic nature.

As disclosed herein, the composition can comprise surfactants present in an amount ranging, for example, from 0.01% to 40% by weight, relative to the total weight of the composition and, for example, from 0.5% to 30% by weight, relative to the total weight of the composition.

The pH of the ready-to-use bleaching and dye composition ranges from 4 to 12, such as from 7 to 11.5 and further, for example, from 8 to 11.

Another aspect of the disclosure is a ready-to-use pasty anhydrous composition for simultaneously bleaching and dyeing human keratin fibers, such as hair.

For the purposes of the disclosure, the term “ready-to-use composition” means that the composition, intended to be applied onto the keratin fibers, is in unmodified form, i.e. it results from the extemporaneous mixing of the paste and the aqueous hydrogen peroxide composition.

The ready-to-use pasty anhydrous composition, according to the present invention, is obtained by mixing, at the time of use, the said pasty composition with an aqueous hydrogen peroxide composition or by mixing, at the time of use, the said pasty composition, itself obtained by mixing an anhydrous bleaching composition A in paste form comprising at least one peroxygenated salt and at least one alkaline agent and a composition B comprising at least one cationic direct dye, with an aqueous hydrogen peroxide composition.

Also, disclosed herein, is a process for simultaneously bleaching and dyeing human keratin fibers, such as hair.

In a first step, of the present disclosure, an anhydrous bleaching and dyeing composition in paste form is mixed, before use, with an oxidizing agent, such as aqueous hydrogen peroxide composition.

Then the mixture obtained is applied to the area of fibers to be treated.

The mixture is then left on the fibers for a sufficient time ranging, for example, from 3 to 60 minutes, such as ranging from 5 to 40 minutes.

Finally, the mixture is removed, such as by mixing with water, followed by washing with a shampoo and then optionally drying.

Another aspect of the disclosure relates to a multi-compartment device or “kit” for bleaching and dyeing human keratin fibers, such as hair, comprising at least two compartments, for example, wherein one of the compartments comprises a pasty anhydrous dye composition according to the invention, and the other comprises an oxidizing agent, such as aqueous hydrogen peroxide composition.

A device may comprise at least two compartments, such as three compartments, wherein one of the compartments comprises a pasty anhydrous bleaching composition A comprising at least one peroxygenated salt, at least one alkaline agent and at least one inert liquid, a second compartment comprises a composition B comprising at least one cationic direct dye, and the third compartment comprises an oxidizing agent, such as aqueous hydrogen peroxide solution.

The following non-limiting examples of simultaneous bleaching and dyeing compositions illustrated the composition disclosed herein.

### EXAMPLE 1

The table below comprises the at least five anhydrous pastes as disclosed herein.

| | Amounts (in g % of starting materials) | | | | | |
|---|---|---|---|---|---|---|
| | A | B | C | D | E | F |
| Potassium persulphate | 30 | 40 | 40 | 40 | 40 | 40 |
| Sodium persulphate | 12 | / | / | / | / | / |
| Sodium disilicate | 7.5 | 7.5 | 7.5 | 7.5 | 7.5 | 7.5 |
| Sodium metasilicate | 6.9 | 6.9 | 6.9 | 6.9 | 6.9 | 6.9 |
| Magnesium carbonate | 1.6 | 3.6 | 5.4 | 3.6 | 3.6 | 3.6 |
| Ammonium chloride | 4.2 | 4.2 | 4.2 | 4.2 | 4.2 | 4.2 |
| EDTA | 1 | 1 | 1 | 1 | 1 | 1 |
| Hexamethyldiisocyanate/ | 2 | 2 | 2 | 2 | 2 | 2 |
| polyethylene glycol | | | | | | |
| copolymer comprising end | | | | | | |
| groups and stearyl- | | | | | | |
| polyoxyethylene, sold | | | | | | |
| under the name Ser-AD | | | | | | |
| FX 1100 by the company | | | | | | |
| Servo Delden | | | | | | |
| carboxymethyl potato | 2 | 2 | 2 | 2 | 2 | 2 |
| starch/weakly crosslinked | | | | | | |
| sodium salt | | | | | | |
| Sodium lauryl sulphate | 4 | 4 | 4 | 4 | 4 | 4 |
| Magnesium stearate | 2 | 2 | 2 | 2 | 2 | 2 |
| Amorphous silica | 1 | 1 | / | / | / | / |
| Hydrogenated silica of | / | / | 0.7 | / | 0.5 | 0.7 |
| hydrophilic nature | | | | | | |
| Hydrogenated polydecene | / | / | / | / | 23.8 | 23.8 |
| sold under the name Silkflo | | | | | | |
| 366 NF Polydecene by the | | | | | | |
| company Amoco Chemical | | | | | | |
| Fumed silica of hydrophobic | / | / | / | 0.5 | 0.5 | / |
| nature | | | | | | |
| Isopropyl myristate | 21.6 | 22.6 | 23.3 | 23.5 | / | / |
| Beeswax | 1.2 | 1.2 | / | / | / | / |
| Basic Red 51 | / | 2 | / | 2 | / | 1.3 |
| Basic Orange 31 | 2 | / | / | / | / | 1 |
| Basic Blue 41 | / | / | 1 | / | / | / |
| Basic Red 22 | / | / | / | / | 2 | / |
| Basic Yellow 67 | / | / | / | 0.8 | / | / |

### EXAMPLE 2

The table below comprises:

composition B,

composition Ba, which was identical to composition B, but comprised no direct dye,

composition G, a pulverulent composition in accordance with the prior art,

composition Ga, a pulverulent composition in accordance with the prior art, but comprised no direct dye.

| | Amounts (in g % of starting materials) | | | |
|---|---|---|---|---|
| | B | | G (pulverulent | |
| | (invention) | Ba | composition) | Ga |
| Potassium persulphate | 40 | 40 | 25 | 25 |
| Sodium persulphate | / | / | 25 | 25 |
| Sodium disilicate | 7.5 | 7.5 | / | / |
| Sodium metasilicate | 6.9 | 6.9 | 12 | 12 |
| Magnesium carbonate | 3.6 | 3.6 | 17.4 | 17.4 |
| Ammonium chloride | 4.2 | 4.2 | / | / |
| Ammonium sulphate | / | / | 4.5 | 4.5 |
| Diammonium phosphate | / | / | 4 | 4 |
| EDTA | 1 | 1 | 2 | 2 |
| Hexamethyldiisocyanate/ | 2 | 2 | / | / |
| polyethylene glycol | | | | |
| copolymer comprising | | | | |
| end groups and | | | | |
| stearyl-polyoxyethylene, | | | | |
| sold under the name | | | | |
| Ser-AD FX 1100 by the | | | | |
| company Servo Delden | | | | |
| carboxymethyl potato | 2 | 2 | / | / |
| starch/weakly | | | | |
| crosslinked sodium salt | | | | |
| Guar gum | / | / | 2 | 2 |
| Sodium lauryl sulphate | 4 | 4 | 3 | 3 |
| Magnesium stearate | 2 | 2 | / | / |
| Silica | 1 | 3 | 3.1 | 5.1 |
| Hydrogenated silica of | / | / | 0.7 | / |
| hydrophilic nature | | | | |
| Isopropyl myristate | 22.6 | 22.6 | / | / |
| Beeswax | 1.2 | 1.2 | / | / |
| Basic Red 51 | 2 | / | 2 | / |

Compositions B, Ba, G and Ga were mixed in a ratio of 1 to 1.5 with an aqueous 12% hydrogen peroxide composition, and the ready-to-use compositions thus formed were then applied onto three locks of chestnut-brown hair with a bath ratio of 10, for a leave-in time of 30 minutes, at a temperature which ranged from 38° C. to 42° C. After treatment, the locks were rinsed with water, shampooed, and then dried.

Ccolorimetric measurements on the three locks of hair were performed.

The bleaching power of compositions Ba and Ga were measured. The lightening was read using a Minolta CM 2002 colorimeter in the CIE L*a*b* international system. Each lightening test was performed three times to be able to evaluate the average color difference, ΔE, and the standard deviation relative to the unbleached chestnut-brown control lock.

The color difference, ΔE, was calculated by applying the following equation:

ΔE=\sqrt{(L*−L₀*)²+(a*−a₀*)²+(b* −b₀*)²}

In this equation, ΔE represented the difference in color between the bleached lock and the control lock, L*, a* and b* respectively represented the measurements for the bleached lock, Lₒ*, aₒ* and bₒ* respectively represented the measurements for the chestnut-brown control lock.

In the table below collating the measurement results, the higher the value of ΔE, the greater the color difference between the two locks, and the greater the lightening.

| | Composition Ba | Composition Ga |
|---|---|---|
| Lightening ΔE | 43.0 | 41.6 |
| | standard deviation 1.7 | standard deviation 2.6 |

These results showed that the lightening or bleaching performance obtained with the paste of the invention was equivalent to that of the prior art.

The dyeing power of compositions B and G were measured. The chromaticity was read using a Minolta CM 2002 calorimeter in the CIE L*a*b* international system and in the TSL (Hue, Saturation, Luminosity) or L*C*h system.

The chromaticity was calculated according to the formula:

C=\sqrt{a*²+b*²}

The results are given in the table below:

| | Composition B | Composition G |
|---|---|---|
| Chromaticity C* | 33.7 | 28.2 |
| | standard deviation 1.2 | standard deviation 1.6 |

The ready-to-use composition obtained from composition B, in accordance with the invention, gave very chromatic red locks. On the other hand, the ready-to-use composition obtained from composition G, in accordance with the prior art, gave less chromatic mahogany-red locks.

These results showed that composition B afforded much better dyeing performance than the performance of the prior art.

What is claimed is:

1. A pasty anhydrous composition, for simultaneously bleaching and dyeing human keratin fibers, comprising, in a medium that is suitable for dyeing:
at least one peroxygenated salt,
at least one alkaline agent,
at least one inert liquid present in an amount ranging from 15% to 35% by weight, relative to the total weight of the composition, and
at least one cationic direct dye.

2. The composition according to claim 1, wherein the at least one cationic direct dye is chosen from xanthene dyes, azo dyes, azomethine dyes and methine dyes.

3. The composition according to claim 1, wherein the at least one cationic direct dye is chosen from heterocyclic direct dyes.

4. The composition according to claim 1, wherein the at least one cationic direct dye comprises at least one cationic charge on a heterocycle.

5. The composition according to claim 1, wherein the at least one cationic direct dye is chosen from azo, azomethine, and methine direct dyes comprising at least one cationic charge on a heterocycle.

6. The composition according to claim 1, wherein the at least one cationic direct dye is chosen from: Acid Red 52, Basic Blue 41, Basic Blue 67, Basic Brown 1, Basic Brown 4, Basic Red 18, Basic Red 22, Basic Red 46, Basic Red 104, Basic Violet 35, Basic Yellow 45, Basic Yellow 57, Basic Yellow 67, Basic Red 14, Basic Yellow 13, Basic Yellow 29, and dyes of formula (I):
G-N=N-J (I)
wherein:
G is chosen from formulae G₁, G₂ and G₃ below: 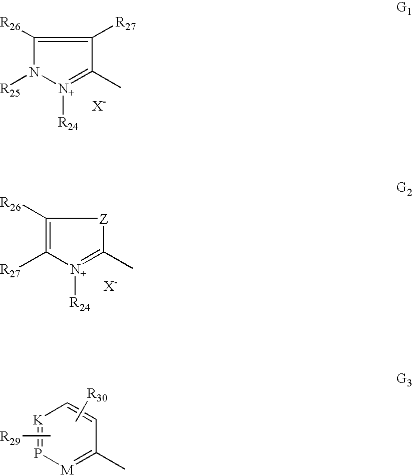
wherein:
R₂₄ is chosen from C₁-C₄ alkyl, phenyl which may be substituted with C₁-C₄ alkyl, and halogen atoms chosen from chlorine, bromine, iodine and fluorine;
R₂₅ is chosen from C₁-C₄ alkyl and phenyl;
R₂₆ and R₂₇, which may be identical or different, are chosen from C₁-C₄ alkyl and phenyl, and additionally wherein R₂₆ and R₂₇ may form together in G₁ a benzene ring comprising at least one substituent chosen from C₁-C₄ alkyl, C₁-C₄ alkoxy, and NO₂, and also wherein R₂₆ and R₂₇ may form together in G₂ a benzene ring optionally comprising at least one substituent chosen from C₁-C₄ alkyl, C₁-C₄ alkoxy, and NO₂;
R₂₆ may also be chosen from hydrogen;
Z is chosen from oxygen, sulphur and groups of formula —NR₂₅;
M is chosen —CH, —CR (wherein R comprises C₁-C₄ alkyl), and —N R₂₈(X⁻)ᵣ;
K is chosen from —CH, —CR (wherein R comprises C₁-C₄ alkyl), and —NR₂₈(X⁻)ᵣ;
P is chosen from —CH, —CR (wherein R comprises C₁-C₄ alkyl), and —NR₂₈(X⁻)ᵣ;
wherein r is an integer chosen from 0 and 1;
R₂₈ is chosen from O⁻, C₁-C₄ alkoxy, and C₁-C₄ alkyl;
R₂₉ and R₃₀, which may be identical or different, are chosen from hydrogen and halogen atoms chosen from chlorine, bromine, iodine and fluorine, C₁-C₄ alkyl,C₁-C₄ alkoxy, and —NO₂;
X⁻ is chosen from anions
J is chosen from:
(a) J₁ below: 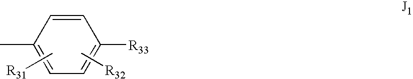
wherein:
R₃₁ is chosen from hydrogen, halogen atoms chosen from chlorine, bromine, iodine and fluorine, C₁-C₄ alkyl, C₁-C₄ alkoxy, —OH, —NO₂, —NHR₃₄, —NR₃₅R₃₆, and C₁-C₄—NHCOalkyl, and wherein R₃₁ may form with R₃₂, at least one ring chosen from 5- and 6-membered rings optionally comprising at least one hetero atom chosen from nitrogen, oxygen and sulphur;
R₃₂ is chosen from hydrogen, halogen atoms chosen from chlorine, bromine, iodine and fluorine, C₁-C₄, C₁-C₄ alkoxy, and wherein R₃₂ may form with R₃₃ and R₃₄, at least one ring chosen from 5- and 6-membered rings optionally comprising at least one hetero atom chosen from nitrogen, oxygen and sulphur;
R₃₃ is chosen from hydrogen, —OH, —NHR₃₄ and —NR₃₅R₃₆;
R₃₄ is chosen from hydrogen, C₁-C₄ alkyl, C₁-C₄ monohydroxyalkyl, C₂-C₄ polyhydroxyalkyl, and phenyl;
R₃₅ and R₃₆, which may be identical or different, are chosen from C₁-C₄ alkyl, C₁-C₄ monohydroxyalkyl, and C₂-C₄ polyhydroxyalkyl;
(b) 5- and 6-membered nitrogenous heterocyclic groups, optionally comprising other hetero atoms and carbonyl groups and optionally comprising at least one substituent chosen from C₁-C₄ alkyl, amino, and phenyl, such as groups of formula J₂ below: 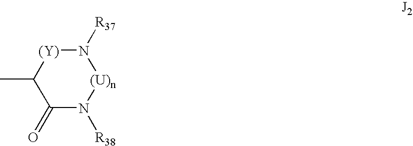
wherein:
R₃₇ and R₃₈, which may be identical or different, are chosen from hydrogen, C₃-C₁₀ alkyl, and phenyl;
Y is chosen from —CO— and —C(CH₃)═;
n is an integer chosen from 0 and 1, wherein when n is equal to 1,U is chosen from —CO—,
compounds of formula (II) below: 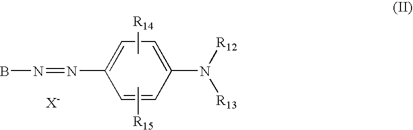
wherein:
R₁₂ is chosen from hydrogen and C₁-C₄ alkyl,
R₁₃ is chosen from hydrogen, alkyl optionally having at least one substituent chosen from —CN, amino, and 4′-aminophenyl, and wherein R₁₃ may form with R₁₂ a heterocycle optionally comprising at least one heteroatom chosen from oxygen and nitrogen, wherein the heterocycle may optionally have at least one substituent chosen from C₁-C₄ alkyl,
R₁₄ and R₁₅, which may be identical or different, are chosen from hydrogen, halogen atoms chosen from bromine, chlorine, iodine and fluorine, C₁-C₄ alkyl, C₁-C₄ alkoxy, and —CN,
X⁻ is chosen from anions
B is chosen from B1 to B6 below: 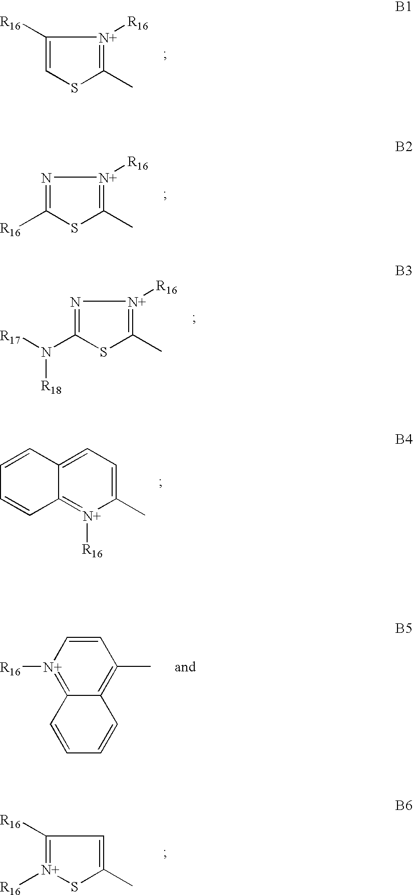
wherein R₁₆, which may be identical or different, is chosen from C₁-C₄ alkyl,
R₁₇ and R₁₈, which may be identical or different, are chosen from hydrogen and C₁-C₄ alkyl,
compounds of formulae (III) and (IV) below: 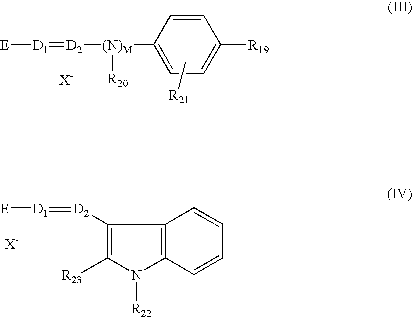
wherein:
R₁₉ is chosen from hydrogen, C₁-C₄, halogen, and amino,
R₂₀ is chosen from hydrogen and C₁-C₄ alkyl, and wherein R₂₀ may form, with a carbon atom of the benzene ring, a heterocycle optionally comprising oxygen and optionally having at least one substituent chosen from C₁-C₄ alkyl,
R₂₁ is chosen from hydrogen and halogen atom,
R₂₂ and R₂₃, which may be identical or different, are chosen from hydrogen and C₁-C₄ alkyl,
D₁ and D₂, which may be identical or different, are chosen from nitrogen and —CH,
m is an integer chosen from 0 and 1,
wherein when R₁₉ is chosen from unsubstituted amino groups, then D₁ and D₂, which may be identical or different, are chosen from —CH and m is an integer equal to 0,
X⁻ is chosen from anions,
E is chosen from E1 to E8 below: 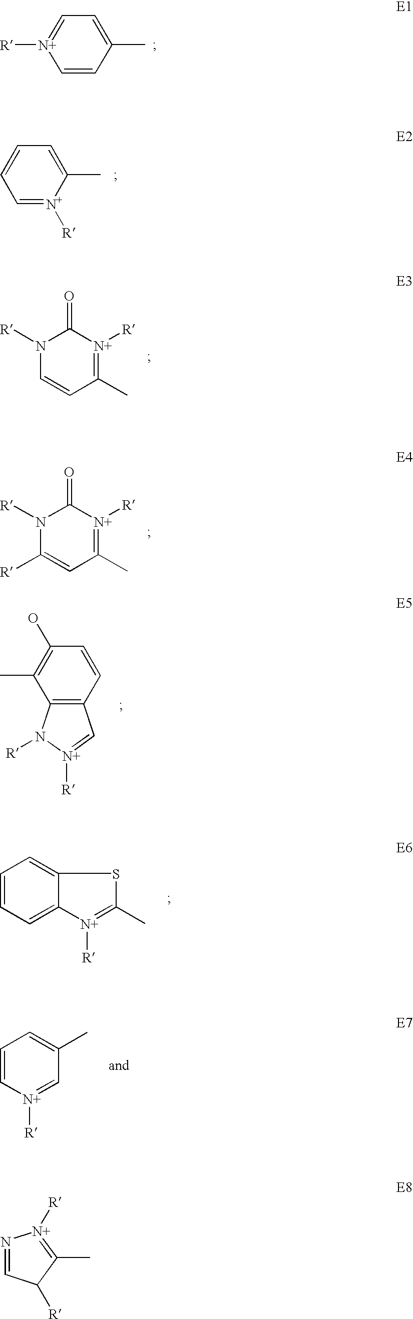
wherein R′ is chosen from C₁-C₄ alkyl;
wherein when m is an integer equal to 0 and D₁ is chosen from nitrogen, then E may be chosen from E9 below: 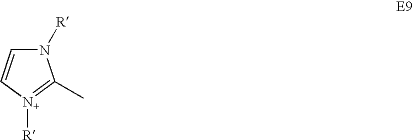
wherein R′, which may be identical or different, is chosen from C₁-C₄ alkyl,
compounds of formula (V) below: 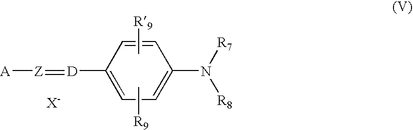
wherein:
Z and D, which may be identical or different, are chosen from nitrogen and —CH—,
R₇ and R₈, which may be identical or different, are chosen from hydrogen; C₁-C₄ alkyl optionally having at least one substituent chosen from —CN, —OH, and —NH₂, and wherein R₇ and R₈ may form, with a carbon atom of the benzene ring, heterocycles optionally comprising oxygen and an additional nitrogen, optionally having at least one substituent chosen from C₁-C₄ alkyl and 4′-aminophenyl,
R₉ and R′₉, which may be identical or different, are chosen from hydrogen, halogen chosen from chlorine, bromine, iodine and fluorine, cyano, C₁-C₄ alkyl, C₁-C₄ alkoxy and acetyloxy,
X⁻ is chosen from anions,
A is chosen from A1 to A19 below: 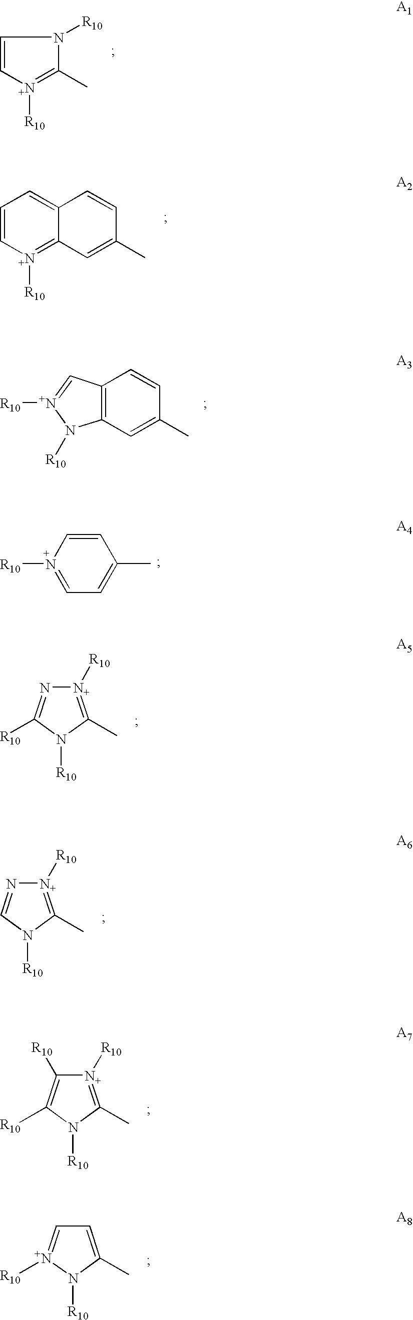 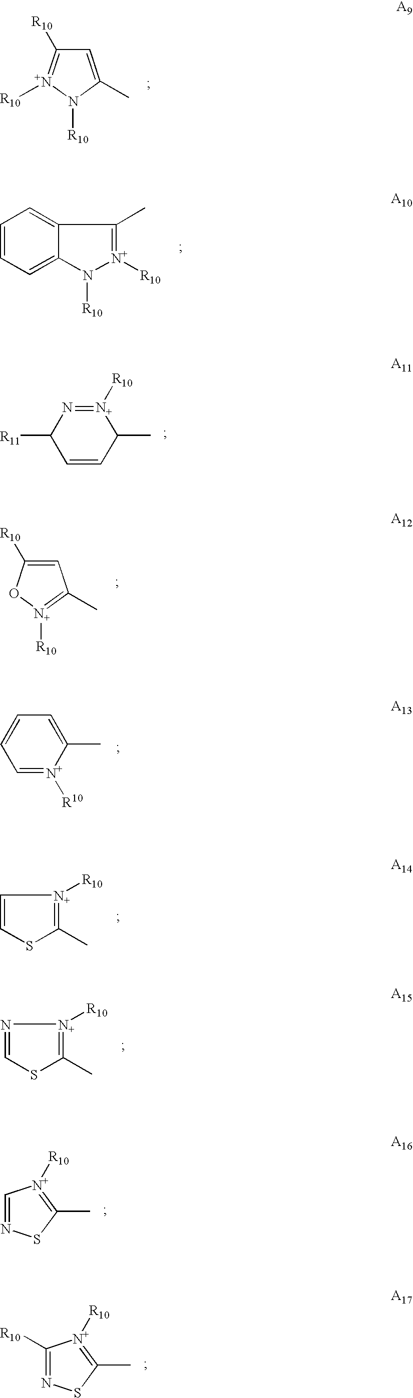 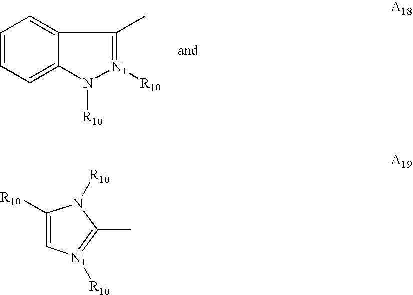
wherein R₁₀, which may be identical or different, is chosen from C₁-C₄ alkyl optionally having at least one hydroxyl substituent and R₁₁ is chosen from C₁-C₄ alkoxy,
compounds of formulae (VI), (VII) and (VIII): 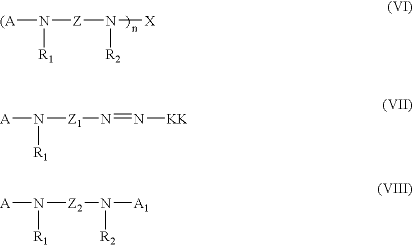
wherein:
A and A₁, which may be identical or different, are chosen from residues of formula: 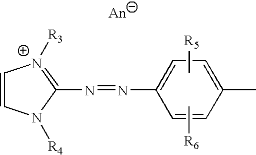
Z is chosen from aliphatic and aromatic diamine residues,
R₁ and R₂, which may be identical or different, are chosen from hydrogen and C₁-C₄ alkyl, and wherein R₁ and R₂ may form, together with two nitrogen atoms to which they are attached and with Z and Z₂,
a ring chosen from 5-, 6- and 7-membered rings,
X is chosen from residues of a chain unit forming a bridge,
n is and integer chosen from 2, 3 and 4,
Z₁ is chosen from residues of aromatic diamines,
Z₂ is chosen from residues of aliphatic diamines,
KK is chosen from residues of a coupling compound,
R₃ and R₄, which may be identical or different, are chosen from hydrogen and C₁-C₄ alkyl groups,
R₅ and R₆, which may be identical or different, are chosen from hydrogen, C₁-C₄ alkyl, and C₁-C₄ alkoxy,
An^{Θ} is chosen from colorless anions.

7. The composition according to claim 1, wherein the at least one cationic direct dye is chosen from Basic Red 51, Basic Yellow 87 and Basic Orange 31.

8. The composition according to claim 1, wherein the at least one cationic direct dye is present in an amount ranging from 0.001% to 20% by weight, relative to the total weight of the composition.

9. The composition according to claim 8, wherein the at least one cationic direct dye is present in an amount ranging from 0.01% to 10% by weight, relative to the total weight of the composition.

10. The composition according to claim 9, wherein the at least one cationic direct dye is present in an amount ranging from 0.1% to 5% by weight, relative to the total weight of the composition.

11. The composition according to claim 1, wherein the at least one peroxygenated salt is chosen from alkali metal and alkaline-earth metal persulphates, perborates, percarbonates, and peroxide.

12. The composition according to claim 1, wherein the peroxygenated salt is chosen from sodium persulphates and potassium persulphates.

13. The composition according to claim 1, wherein the concentration of the at least one peroxygenated salts is present in an amount ranging from 10% to 70% by weight, relative to the total weight of the composition.

14. The composition according to claim 13, wherein the at least one peroxygenated salt is present in an amount ranging from 20% to 60% by weight, relative to the total weight of the composition.

15. The composition according to claim 1, wherein the at least one alkaline agent is chosen from urea, ammonium chlorides, ammonium sulphates, ammonium phosphates, ammonium nitrates, silicates, and alkali metal and alkaline-earth metal phosphates, and carbonates.

16. The composition according to claim 1, wherein the at least one alkaline agent is present in an amount ranging from 0.01% to 40% by weight, relative to the total weight of the composition.

17. The composition according to claim 16, wherein the at least one alkaline agent is present in an amount ranging from 0.1% to 30%, by weight, relative to the total weight of the composition.

18. The composition according to claim 1, wherein the at least one inert organic liquid is chosen from groups formed by polydecenes of formula C₁₀ₙH_{[(20n)+2]} wherein n is an integer ranging from 3 to 9, esters of fatty alcohols, esters of fatty acids, esters and diesters of sugar of C₁₂-C₂₄ fatty acids, cyclic ethers, cyclic esters, silicone oils, mineral oils and plant oils.

19. The composition according to claim 18, wherein n is an integer ranging from 3 to 7.

20. The composition according to claim 18, wherein the at least one inert organic liquid is chosen from groups formed by polydecenes of formula C₁₀ₙH_{[(20n)+2]} wherein n is an integer ranging from 3 to 9, esters of fatty alcohols, and esters of fatty acids.

21. The composition according to claim 20, wherein n is an integer ranging from 3 to 7.

22. The composition according to claim 1, wherein the composition comprises at least one nonionic and anionic amphiphilic polymer comprising at least one fatty chain.

23. The composition according to claim 22, wherein the at least one nonionic and/or anionic amphiphilic polymer comprising at least one fatty chain is present in an amount ranging from 0.01% to 30% by weight, relative to the total weight of the composition.

24. The composition according to claim 23, wherein the at least one nonionic and/or anionic amphiphilic polymer comprising at least one fatty chain is present in an amount ranging from 0.1% to 15% by weight, relative to the total weight of the composition.

25. The composition according claim 1, wherein the composition comprises beeswax as an adjuvant.

26. The composition according to claim 1, wherein the composition is a ready-to-use pasty anhydrous composition for simultaneously bleaching and dyeing human keratin fibers, and wherein the composition further comprises hydrogen peroxide.

27. The composition according to claim 26, wherein the pasty anhydrous composition comprises a mixture of anhydrous bleaching composition A in paste form comprising the at least one peroxygenated salt, the at least one alkaline agent and the at least one inert organic liquid, and a composition B comprising the at least one cationic direct dye.

28. A process for simultaneously bleaching and dyeing keratin fibers, comprising:
mixing, before use, an anhydrous bleaching and dyeing composition in paste form, comprising, in a medium that is suitable for dyeing:
at least one peroxygenated salt,
at least one alkaline agent,
at least one inert liquid present in an amount ranging from 15% to 35% by weight, relative to the total weight of the composition, and
at least one cationic direct dye with an aqueous hydrogen peroxide composition,
applying the mixture obtained to the area of fibers to be treated,
leaving the mixture on the fibers for a sufficient time , and
removing the mixture.

29. The process according to claim 28, wherein the sufficient time is from 3 to 60 minutes, and the mixture is removed by mixing with water, followed by washing with a shampoo, and then optionally drying.

30. The process according to claim 29, wherein the sufficient time is from 5 to 40 minutes.

31. A multi-compartment device or multi-compartment “kit” for bleaching and dyeing human keratin fibers, comprising at least two compartments wherein the first compartment comprises a pasty anhydrous composition, comprising, in a medium that is suitable for dyeing:
at least one peroxygenated salt,
at least one alkaline agent,
at least one inert liquid present in an amount ranging from 15% to 35% by weight, relative to the total weight of the composition, and
at least one cationic direct dye, and the second compartment comprises an oxidizing agent.

32. The multi-compartment device or multi-compartment “kit” for bleaching and dyeing human keratin fibers according to claim 31, wherein said oxidizing agent is an aqueous hydrogen peroxide composition.

33. The multi-compartment device or multi-compartment “kit” for bleaching and dyeing human keratin fibers according to claim 31, wherein said human keratin fibers are hair.

34. A multi-compartment device or multi-compartment “kit” for bleaching and dyeing human keratin fibers, comprising at least three compartments wherein the first compartment comprises a pasty anhydrous bleaching composition A comprising at least one peroxygenated salt, at least one alkaline agent, and at least one inert organic liquid, the second compartment comprises a composition B comprising at least one cationic direct dye, and the third compartment comprises an oxidizing agent.

35. The multi-compartment device or multi-compartment “kit” for bleaching and dyeing human keratin fibers according to claim 34, wherein said oxidizing agent is an aqueous hydrogen peroxide composition.

36. The multi-compartment device or multi-compartment “kit” for bleaching and dyeing human keratin fibers according to claim 34, human keratin fibers are hair.

37. The composition according to claim 6, wherein in compounds of formula (II), said anions defining X⁻ are chosen from chloride, methyl sulphates and acetates.

38. The composition according to claim 6, wherein in compounds of formula (I), said anions defining X⁻ are chosen from chloride, iodide, methyl sulphates, ethyl sulphates, acetates and perchlorates.

39. The composition according to claim 6, wherein in compounds of formulae (III) and (IV), in R₁₉ and in R₂₁, halogen is chosen from bromine, chlorine, iodine, and fluorine.

40. The composition according to claim 6, wherein in compounds of formulae (III) and (IV), X⁻ is chosen from chloride, methyl sulphates and acetates.

41. The composition according to claim 6, wherein in compounds of formula (V), X⁻ is chosen from chloride, methyl sulphates and acetates.
